(19) United States
(12) Patent Application Publication
(10) Pub. No.: US 2010/0297697 A1
(43) Pub. Date: Nov. 25, 2010

(54) METHODS FOR INCREASING PROTEIN TITERS

(75) Inventors: Dorothee Ambrosius, Laupheim (DE); Barbara Enenkel, Warthausen (DE); Christian Eckermann, Biberach (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: BOEHRINGER INGELHEIM PHARMA GMBH & CO KG, Ingelheim am Rhein (DE)

(21) Appl. No.: 12/675,218

(22) PCT Filed: Aug. 28, 2008
(86) PCT No.: PCT/EP2008/061310

§ 371 Date: Jun. 11, 2010

(30) Foreign Application Priority Data
Aug. 29, 2007 (EP) 07115227.6

(51) Int. Cl. C12P 21/00; C12N 15/79; C12N 5/10; C07K 14/00; C07K 16/00

(52) U.S. Cl. 435/69.1; 435/320.1; 435/325; 530/350; 530/387.3; 435/326; 435/328; 435/348; 435/254.2; 435/419; 435/349

(57) ABSTRACT

The invention relates to methods of increasing the titre of a protein of interest in a cell as well as the improved production and purification of optimised biomolecules, one component of which is the domain C_{H}3. A frequently observed effect in biomolecules is the cleaving of the C-terminal amino acid(s), e.g. the C-terminal lysine. The usually incomplete processing of the heavy chain of antibodies for example leads to product heterogeneity. To prevent this product heterogeneity the corresponding codon of the C-terminal lysine of the heavy antibody chain has been deleted by recombinant DNA technology. These optimised antibodies lead to a product titre which is higher than in the wild-type. In addition, they prove advantageous during purification by having better elution characteristics as a result of the reduced charge heterogeneity.

### BACKGROUND TO THE INVENTION

1. Technical Field

The invention relates to optimised proteins, particularly antibody Fc fragments, or Fc fusion proteins and methods for the preparation or biopharmaceutical production of those optimised antibodies and Fc fusion proteins with enhanced activity as well as a method of producing and purifying proteins, in which the biomolecule produced is totally homogeneous in relation to the C-terminal lysine.

2. Background

Biomolecules such as proteins, polynucleotides, polysaccharides and the like are increasingly gaining commercial importance as medicines, as diagnostic agents, as additives to foods, detergents and the like, as research reagents and for many other applications. The need for such biomolecules can no longer normally be met—for example in the case of proteins—by isolating molecules from natural sources, but requires the use of biotechnological production methods.

The biotechnological preparation of proteins typically begins with the isolation of the DNA that codes for the desired protein, and the cloning thereof into a suitable expression vector. After transfection of the recombinant expression vectors into suitable prokaryotic or eukaryotic expression cells and subsequent selection of transfected, recombinant cells the latter are cultivated in fermenters and the desired protein is expressed. Then the cells or the culture supernatant is or are harvested and the protein contained therein is worked up and purified.

In the case of biopharmaceuticals, such as for example proteins used as medicaments, e.g. therapeutic antibodies, the yield of product is critical. The separation of impurities is also important. A distinction may be drawn between process- and product-dependent impurities. The process-dependent impurities contain components of the host cells such as proteins and nucleic acids or originate from the cell culture (such as media constituents) and from the working up (such as for example salts or dissolved chromatography ligands). In addition, product-dependent impurities also occur. These are molecular variants of the product with different properties. These include for example abbreviated forms such as precursors and hydrolytic breakdown products, enzymatic cleaving of C-terminal amino acid groups of proteins, but also modified forms, produced for example by deamination, different glycosylation patterns or wrongly linked disulphide bridges The product-dependent variants include polymers and aggregates. The term contaminants refers to all other materials of a chemical, biochemical or microbiological nature which do not belong directly to the manufacturing process. Further contaminants are for example viruses which may occur undesirably in cell cultures.

A frequently observed product variant in the overexpression of recombinant antibodies or Fc fusion proteins in mammals for the production of new biopharmaceutical medicines is based on the heterogeneity at the C-terminus of the heavy chain of immunoglobulins by enzymatic cleaving of the C-terminal lysine. To describe this heterogeneity exactly, high-resolution analytical methods have to be developed. For the detection and quantification of the charge heterogeneity, the following methods are used in quality control in the pharmaceutical industry: Cation Ion Exchange chromatography (CIEX), isoelectric focusing (IEF) (detection only), capillary isoelectric focusing (cIEF) and Liquid Chromatography Mass Spectrometry (LCMS). Each batch produced has to be evaluated and passed in respect of this modification, inter alia.

In many of the molecules of this category that are on the market, these product heterogeneities at the C-terminus of the heavy chain are observed. A distinction is made between antibody monomers with a fully cleaved lysine (Lys0), with a cleaved lysine (Lys1) and without lysine cleaving (Lys2) at the C-terminus of the heavy chain. The different incompletely processed molecules (Lys1 and Lys2) may account for up to 30% within a charge (Santora et al. (1999) Analytical Biochemistry, 275(1): p. 98-108). In the process for manufacturing Remicade® (Infliximab) the heterogeneity during the fermentation was approx. 20% (Lys0 and Lys1) and 80% (Lys2) (FDA, Product Review on Remicade, 1998). Further examples of C-terminal lysine processing in monoclonal antibodies can be found in the Table (Harris, R. J., (1995) Journal of Chromatography A, 705 (1), pp. 129-134).

| Protein | Amino acid | Cell line/Source | Reference |
|---|---|---|---|
| rCD4-IgG | Lys | transfected CHO | R. J. Harris, K. L. Wagner and M. W. Spellman, |
| | | | Eur. J. Biochem., 194 |
| | | | (1990) 611-620 |
| rhu MabHER2 | Lys | transfected CHO | R. J. Harris, A. A. Murnane, S. L. Utter, |
| | | | K. L, Wagner, E. T. Cox, G. Polastri, |
| | | | J. C. Helder and M. B. Sliwkowski, |
| | | | Bio/Technology, 11 |
| | | | (1993) 1293-1297 |
| OKT3 Mab | Lys | hybridoma | P. Rao, A. Williams, A. Baldwin- |
| | | | Ferro, E. Hanigan, D. Kroon, M. Makowski, |
| | | | E. Meyer, V. Numsuwan, |
| | | | E. Rubin and A. Tran, BioPharm, 4 |
| | | | (1991) 38-43. |
| OKT3 Mab | Lys | hybridoma | P. Rao, A. Williams, A. Baldwin- |
| | | | Ferro, E. Hanigan, D. Kroon, M. Makowski, |
| | | | E. Meyer, V. Numsuwan, |
| | | | E. Rubin and A. Tran, BioPharm, 4 |
| | | | (1991) 38-43. |
| CEM231 Mab | Lys | hybridoma | J. P. McDonough, T. C. Furman, R. M. Bartholomew |
| | | | and R. A. Jue, U.S. Pat. No. |
| | | | 5,126,250 (1992) |
| CEM231 Mab | Lys | hybridoma | J. P. McDonough, T. C. Furman, R. M. Bartholomew |
| | | | and R. A. Jue, U.S. Pat. No. |
| | | | 5,126,250 (1992) |
| Hu-anti-Tac | Lys | transfected SP2/0 | D. A. Lewis, A. W. Guzzetta, W. S. Hancock |
| | | | and M. Costello, Anal. |
| Mab | | | Chem., 66 (1994) 585-595. |
| 2-Chain tPA | Arg | transfected CHO | |
| 2-Chain tPA | Arg | melanoma | |
| hu EPO | Arg | human urine | M. A. Recny, H. A. Scoble and Y. Kim, |
| | | | J. Biol. Chem., 262 (1987) 17156-17163 |
| rhu EPO | Arg | transfected CHO | M. A. Recny, H. A. Scoble and Y. Kim, |
| | | | J. Biol. Chem., 262 (1987) 17156-17163 |

Source: Harris, R. J. (1995) Journal of Chromatography A, 705 (1), pp. 129-134

The cause of this product heterogeneity is not known at present. It is unclear whether the structure of the chain, the host cell or the fermentation conditions and hence different metabolic processes in the cell have a major influence. It is also currently unknown at which point in the manufacture of the product in the cell (co-translational, post-translational), where and by means of which carboxypetidase the cleaving of the lysine is carried out. Possible variations between batches may therefore not be prevented and targeted counter-control is thus not possible.

Product heterogeneities may also be caused by other C-terminal amino acid deletions, such as e.g. by a deletion of the C-terminal arginine at the proteins tPA or EPO (Harris, R. J., (1995) Journal of Chromatography A, 705 (1), pp. 129-134).

The starting point for evaluating the production batch is the physicochemical product qualities, the purity, homogeneity and effectiveness and safety of the product.

Electrophoretic (IEF) or chromatographic (IEC, SEC, RP) separation methods and mass-spectroscopic processes (MS, ESI, MALDI) are used to evaluate the purity and heterogeneity of the product.

Monitoring product purity ensures an adequate elimination of impurities and the removal of cleavage products and aggregated protein molecules formed by enzymatic, mechanical or chemical processes. The product homogeneity is evaluating primarily by means of the deviations in the glycosylation pattern and the charge heterogeneity. The effectiveness of a product describes its biological activity, which in the case of antibodies is made up of properties such as its antigen binding capacity, the induction of effector functions, serum half-life and so on. Determining factors for product safety include inter alia the sterility and bacterial endotoxin load of the batch of product.

Because of the number of control values that have to be guaranteed or adhered to in a production batch to enable it to be released, a reduction in the control values, e.g. by eliminating the parameter affecting the batch, is desirable.

Moreover, in the biotechnological preparation of proteins a high product titre and a high specific productivity of the cells is desirable.

The problem thus arises of providing an improved manufacturing process. With regard to product expression, product purification and product stability, no negative influences should occur during manufacture.

The present invention surprisingly solves this problem with a process for preparing proteins which makes it possible to obtain an increased yield, by removing the C-terminally coding codon (e.g. lysine) at the DNA level and then inserting a stop codon.

This process makes it possible to increase the protein titre, particularly of antibodies, which have a C-terminal lysine deletion on the heavy chains.

### SUMMARY OF THE INVENTION

The present invention describes recombinant DNA constructs of proteins, particularly antibody molecules such as IgG1, IgG2, IgG3, IgG4 and Fc fusion constructs which comprise a deletion of the C-terminal lysine. This change to the expression construct and the deletion of the C-terminal Lys codon means that only molecules with a homogeneous C-terminus of the heavy chain are prepared.

In the overexpression of for example recombinant antibodies or Fc fusion proteins in mammalian cells for preparing new biopharmaceutical medicaments molecules often occur which have heterogeneities at the C-terminus of the heavy chain. The purified end product has three different species with regard to the C-terminus of the heavy chain: 1) complete chains with C-terminal lysine according to the DNA sequence (Lys 2) or 2) incomplete chain (Lys 1) and 3) deletion of the C-terminal lysine on both chains (Lys 0). The proportions of the two species are unpredictable. Thus, differences may occur depending on the cell, fermentation conditions and manufacturing batch. It is unclear whether the antibody structure influences this intracellular enzymatic cleaving of the lysine.

The procedure with the molecules on the market up till now was to express the complete DNA sequence of the heavy chain and analyse and document any heterogeneities occurring at the C-terminus at great expense. Thus, in order to characterise the product, accurate methods of analysing the C-terminus have to be developed and all the batches have to be analysed with regard to this feature (Alexandru C. Lazar et al; Rapid Commun. Mass Spectrum. 2004; 18: 239-244, Lintao Wang et al; Pharmaceutical Research, Vol. 22, No. 8, 2005). The cost of analysing the product heterogeneities occurring is therefore considerable. It would be desirable to reduce the effort and expenditure of analysis.

The cause of the product heterogeneity described is not known at present. It is unclear whether the structure of the chain, the host cell or the fermentation conditions and hence different metabolic processes in the cell have a major influence. It is also currently unknown at which point in the manufacture of the product in the cell (co-translational, post-translational), where and by which enzyme the cleaving of the lysine is carried out.

Possible fluctuations between batches may therefore not be prevented and targeted counter-control is thus not possible.

Hitherto there has been no indication that the use of these products gives rise to any detrimental effects such as e.g. immunopathological side effects as a result of the heterogeneity in the C-terminus. Therefore the non-native sequence without C-terminal lysine also appears to be acceptable in terms of clinical efficacy and tolerance and to be equivalent to the native sequence.

Up till now, however, no constructs for therapeutic proteins, particularly antibodies or Fc fusion constructs, with the deletion of the C-terminal Lys codon have been described, as the C-terminal lysine in the heavy chain of IgGs is highly conserved.

In a departure from the prior art, in the present invention the codon for lysine in the expression construct for the heavy chain of antibodies has already been deleted at the DNA level at the 3′ end. In all the IgG subtypes the C-terminus of the heavy chain is highly conserved and the lysine at the C-terminus is always present for example both in human and in murine antibodies. In view of this situation it is to be expected that the lysine is of particular importance for the expression, folding or secretion. Surprisingly, however, our experiments with different categories of IgG showed for the first time that in spite of the deletion of the C-terminal lysine the molecules are expressed in animal cell culture systems and the native protein structure is secreted into the medium. A particularly surprising aspect is the totally unexpected increase observed in the product titre when these constructs are used. This is unexpected in view of the high conservation of the C-terminal lysine position in the preferably human immunoglobulins. The product titre is increased by at least 10%, preferably by at least 20% and particularly preferably by at least 50% when these expression constructs are used.

It has also been possible to provide qualitative and quantitative evidence of the avoidance of product heterogeneity as a result of the deletion of the Lys codon by analysing the antibody isotypes produced. For two different isotypes (IgG1 and IgG4) the wild-type antibody and the corresponding lysine deletion mutant were expressed and purified as a comparison. Then the protein characterisation was carried out. Moreover, contrary to expectations, it was shown that the product titre could be increased by at least 10-20%. In the working up of the product (protein A affinity chromatography) and the protein characterisation the deletion of the lysine codon was not found to have any harmful effects. Further analysis of the purification process and product characterisation (product yield, aggregation characteristics) once again the Lys deletion mutants were not found to have any negative influence. In view of the greater robustness for the manufacturing process, the reduction in the analytical work and the increased product titre this new method is clearly superior to the prior art.

The chief advantage over the current prior art is that when these constructs are used only the variant of the C-terminus without lysine can occur for the heavy chain. Thus there is no possibility of fluctuations between batches and the amount of product characterisation work. A particularly surprising of the present invention is that the constructs without C-terminal lysine lead to increased product titres, which is particularly advantageous for a high yield.

The present invention may preferably be applied to processes for preparing recombinant antibodies and/or Fc fusion proteins. The present invention may, however, also be applied to other molecules that comprise C-terminal amino acid deletions. Examples of these are EPO and tPA in which C-terminal arginine deletions occur.

The invention relates to the improved production and purification of optimised proteins, one ingredient of which is, inter alia, the immunoglobulin domain C_{H}3. A frequently observed effect of these proteins is the cleaving of the C-terminal lysine. This usually incomplete processing of the heavy chain leads to product heterogeneity. In order to avoid this product heterogeneity the corresponding codon of the C-terminal lysine of the heavy antibody chain was deleted by recombinant DNA technology. This deletion in the optimised antibody surprisingly results not in a disadvantage in the expression or intracellular protein processing, but in an increased product titre compared with the wild- type. In addition, the optimised antibodies have proved to be advantageous in purification by a better elution process on account of the reduced charge heterogeneity and are characterised by an improved homogeneity. Another advantage is that in the purification of the protein of interest a lower salt concentration is used compared with the purification of a protein without the deletion of the C-terminal amino acid.

The present invention does not arise from the prior art. At present the product heterogeneity has to be analysed for each production batch before it can be released. Labour-intensive and high-cost methods of analysis have to be used for the qualitative and quantitative determination of the heterogeneity of lysine groups at the C-terminus of the heavy chain.

Established methods used for the quantitative determination of the antibody isoforms are the methods used in quality control in the pharmaceutical industry, such as column chromatographical methods of separation (weak cation exchangers, WCX), sometimes in conjunction with mass spectroscopy (LC-MS) or electrophoretic separation methods (capillary isoelectric focussing, CIEF). Gel-isoelectrophoretic focussing only permits qualitative evaluation of the lysine heterogeneity.

One approach to reducing charge heterogeneity by means of C-terminal lysine groups of the heavy antibody chain is described in existing methods of reducing the heterogeneity of monoclonal antibodies (EP0361902, U.S. Pat. No. 5,126,250). A reduction in heterogeneity is achieved here by different methods, such as the lowering of the pH, the enzymatic cleaving of C-terminal lysine groups by carboxypeptidase or the addition of ascites liquid.

In the enzymatic process the reduction in charge heterogeneity is obtained by the cleaving of C-terminal lysine groups of the heavy chain of immunoglobulin antibodies by means of the enzyme carboxypeptidase. This process however achieves only a conversion of 95% of the antibodies into the homogeneous antibody form (Lys0). Other methods consist in the incubation of the heterogeneous antibody forms with ascites fluid in different ratios (2:1 to 1:10) or in a reduction in the pH of the culture medium. The efficiency of these methods of C-terminal lysine cleaving is also only approx. 95%. All the processes are also time-consuming (>24h).

### DESCRIPTION OF THE FIGURES

FIG. 1: SCHEMATIC REPRESENTATION OF THE RECOMBINANT VECTORS

The vectors shown here are used for the expression of the monoclonal antibodies of IgG1- and IgG4-isotype in CHO-DG44 cells. “P/E” denotes a combination of CMV-enhancer and hamster Ub/S27a-promoter, “CMV” denotes a combination of CMV-enhancer and -promoter, “P” merely denotes a promoter element and “T” a termination signal for the transcription, which is required for the polyadenylation of the transcribed mRNA. The position and direction of the transcription initiation within each transcription unit is indicated by an arrow. The amplifiable selectable marker dihydrofolate-reductase is abbreviated to “dhfr”. The selectable marker neomycin-phosphotransferase is designated “npt” and the neomycin phosphotransferase mutant produced by point mutation F240I is referred to as “npt F240I”. “IgG1 HC” codes for the heavy chain of the wild-type F19-antibody of the IgG1 isotype and “IgG1-Lys” for the heavy chain of this antibody with a C-terminal lysine deletion. “IgG4 HC” denotes the gene for the heavy chain of the IgG4-wild-type and “IgG4-Lys” in turn denotes the heavy chain of the IgG4 with a C-terminal lysine deletion. “LC” codes for the light chain of the IgG1- or IgG4-antibody.

FIG. 2: INFLUENCE OF THE C-TERMINAL LYSINE DELETION ON THE TRANSIENT EXPRESSION OF AN IGG1-ANTIBODY

In order to check whether the conserved C-terminal Lysine of the heavy chain has an influence on the expression or secretion of the IgG1 molecule, a co-transfection of CHO-DG44 cells with the plasmid combinations pBID/F19HC and pBIN/F19LC (IgG1 with C-terminal lysine, cross-hatched bar) or BID/IgG1-Lys and pBIN/F19LC (IgG1 with C-terminal lysine deletion, dotted bar) is carried out. At the same time a SEAP expression plasmid (=secreted alkaline phosphatase) is co-transfected in order to compare the transfection efficiency. 48 h after transfection the cell culture supernatants are removed and the IgG1 titre is determined by ELISA and the SEAP activity is measured. The IgG1-titre is corrected with regard to the transfection efficiency. The Figure shows the average of 10 parallel pools in each case with comparable amounts of product for both variants.

FIG. 3: EXPRESSION OF IGG1-WILD-TYPE AND IGG1-LYSINE-DELETION MUTANT IN STABLE UNAMPLIFIED CELL POOLS

In stably transfected cells the influence of the C-terminal lysine deletion on the expression of an IgG1 antibody is investigated. For this, CHO-DG44 cells are transfected with the plasmid combinations pBID/F19HC and pBIN/F19LC (IgG1 with C-terminal lysine=IgG1-WT) or BID/IgG1-Lys and pBIN/F19LC (IgG1 with C-terminal lysine deletion=IgG1-Lys). After a two- to three-week selection of the transfected cell pools, in each case 10 per plasmid combination, in hypoxanthine/thymidine-free medium with the addition of G418, the concentration of the IgG1 antibody produced in the cell culture supernatant is determined by ELISA and the specific productivity per cell and per day is calculated. The bars represent the mean values of the specific productivity (dotted bar) or of the titre (striped bar) of all the pools in the test consisting of in each case 3-4 cultivation passages in 75 cm² cell culture flasks.

FIG. 4: EXPRESSION OF IGG1-WILD-TYPE AND IGG1-LYSINE DELETION MUTANT IN STABLE AMPLIFIED CELL POOLS

CHO-DG44 cells are transfected with the plasmid combinations pBID/F19HC and pBIN/F19LC (IgG1 with C-terminal lysine=IgG1-wild-type) or BID/IgG1-Lys and pBIN/F19LC (IgG1 with C-terminal lysine deletion=IgG1-lysine). After a two- to three-week selection of the transfected cell pools (in each case 10 pools per plasmid combination) in hypoxanthine/thymidine-free medium with the addition of G418 a DHFR-mediated gene amplification is then carried out by adding 100 nM methotrexate (MTX) to the cultivation medium. The concentration of the IgG1 antibody produced in the cell culture supernatant is determined by ELISA and the specific productivity per cell and per day is calculated. The bars represent on the one hand the mean values of the specific productivity (dotted bar) or of the titre (striped bar) of each individual pool in the test each comprising 6 cultivation passages in 75 cm² cell culture flasks. On the other hand the mean value (MW) of all the pool data is also given.

FIG. 4A shows the data of the cells pools transfected with the IgG1 wild-type, while FIG. 4B shows the data of the cells pools transfected with the IgG1-lysine-deletion variant. The latter produce on average 86% more antibodies at 120% higher specific productivity than the cell pools transfected with the IgG1-wild-type.

FIG. 5: INFLUENCE OF THE C-TERMINAL LYSINE DELETION ON THE TRANSIENT EXPRESSION OF AN IGG4-ANTIBODY

In order to check whether the conserved C-terminal lysine of the heavy chain has an influence on the expression or secretion of the IgG4 molecule, a co-transfection of CHO-DG44 cells with the plasmid combinations pBIDa/IgG4 HC and pBIN8a/IgG4 LC (IgG4 with C-terminal lysine, cross-hatched bar) or BIDa/IgG4-Lys and pBIN8a/IgG4 LC (IgG4 with C-terminal lysine deletion, dotted bar) is carried out. At the same time a SEAP expression plasmid (=secreted alkaline phosphatase) is co-transfected in order to compare the transfection efficiency. 48 h after transfection the cell culture supernatants are removed and the IgG4 titre is determined by ELISA and the SEAP activity is measured. The IgG4 titre is corrected with regard to the transfection efficiency. The Figure shows the mean value of 10 parallel pools in each case with even somewhat higher product titres of the IgG4-antibody with C-terminal lysine deletion.

FIG. 6: EXPRESSION OF IGG4-WILD-TYPE AND IGG4-LYSINE DELETION MUTANT IN STABLE AMPLIFIED CELL POOLS

CHO-DG44 cells are transfected with the plasmid combinations pBIDa/IgG4 HC and pBIN8a/IgG4 LC (IgG4 with C-terminal lysine=IgG4-wild-type) or BIDa/IgG4-Lys and pBIN8a/IgG4 LC (IgG4 with C-terminal lysine deletion=IgG4-lysine). After a two- to three-week selection of the transfected cell pools (in each case 10 pools per plasmid combination) in hypoxanthine/thymidine-free medium with the addition of G418 a DHFR-mediated gene amplification is then carried out by adding 100 nM methotrexate (MTX) to the cultivation medium, thus obtaining successfully amplified cell pools for the IgG4-wild-type 4 and for the IgG4-lysine deletion variant 6. The concentration of the IgG4 antibody produced in the cell culture supernatant is determined by ELISA and the specific productivity per cell and per day is calculated. The bars represent on the one hand the mean values of the specific productivity (dotted bar) or of the titre (striped bar) of each individual pool in the test, each comprising 6 cultivation passages in 75 cm² cell culture flasks. On the other hand the mean value (MW) of all the pool data is also given.

FIG. 6A shows the data of the cells pools transfected with the IgG4 wild-type, while FIG. 6B shows the data of the cells pools transfected with the IgG4-lysine-deletion variant. The latter produce on average 63% more antibodies at 70% higher specific productivity than the cell pools transfected with the IgG4-wild-type.

FIG. 7: EXPRESSION OF IGG4-WILD-TYPE AND IGG4-LYSINE-DELETION MUTANT IN CELL POOLS AFTER A SECOND ROUND OF GENE AMPLIFICATION

CHO-DG44 cells are transfected with the plasmid combinations pBIDa/IgG4 HC and pBIN8a/IgG4 LC (IgG4 with C-terminal lysine=IgG4-wild-type) or BIDa/IgG4-Lys and pBIN8a/IgG4 LC (IgG4 with C-terminal lysine deletion=IgG4-lysine). First of all, a two to three week selection of the transfected cell pools is carried out (in each case 10 pools per plasmid combination) in hypoxanthine/thymidine-free medium with the addition of G418. Then a stepwise DHFR-mediated gene amplification is carried out. In the first step 100 nM methotrexate (MTX) is added to the cultivation medium. With these stable cell pools resulting from this gene amplification, a second round of gene amplification is carried out by adding 400 nM of MTX to the cultivation medium. 6 successfully amplified cell pools are obtained for the IgG4-wild-type 4 and for the IgG4-lysine deletion variant. The concentration of the IgG4 antibody produced in the cell culture supernatant is determined by ELISA and the specific productivity per cell and per day is calculated. The bars represent on the one hand the mean values of the specific productivity (dotted bar) or of the titre (striped bar) of each individual pool in the test, comprising in each case 4 cultivation passages in 75 cm² cell culture flasks. On the other hand the mean value (MW) of all the pool data is also given.

FIG. 7A shows the data of the cells pools transfected with the IgG4 wild-type, while FIG. 7B shows the data of the cells pools transfected with the IgG4-lysine-deletion variant. The latter produce on average 53% more antibodies at 66% higher specific productivity than the cell pools transfected with the IgG4-wild-type.

FIG. 8: QUANTIFICATION OF THE PRODUCT YIELD BY PROTEIN A HPLC

The values determined for the product yield of IgG1 and IgG4 I are over 90% irrespective of the lysine deletion. The proportion of monomer in the isotypes and the corresponding lysine deletion variants is in the range from 89.23 to 97.93%. Both the yield and the monomer content are higher with IgG1-Lys as than with the WT variant.

FIG. 9: ISOELECTRIC FOCUSING (IEF) OF THE ISOTYPES IGG1 AND IGG4

The antibodies were incubated in vitro with carboxypeptidase B in order to cleave any C-terminal lysine present. The isotype IgG1 (+lysine) (=IgG1-wild-type) has a smaller number of protein bands after incubation with carboxypeptidase B (cleaving of the C-terminal lysines at Lys2 and Lys1=>Lys0). The isotype IgG1 (−lysine) (=C-terminal lysine deletion variant) has an identical band pattern independently of the carboxypeptidase B incubation. IEF marker bands can be found at 8.8 kDa and 8.6 kDa.

FIG. 10: DETECTION OF C-TERMINAL LYSINE BY LC-MS

For the isotype IgG4 (+lysine) (=IgG4-wild-type) the proportion of heavy chain (HC) with C-terminal lysine is 20% (light-grey bar HC with lysine), in the Variant IgG4 (−lysine) (=C-terminal lysine deletion variant) it is 0%. The dark-grey bars represent the proportions of heavy chains (HC) without lysine.

FIG. 11: SEPARATION OF THE ANTIBODIES BY WCX

Separation of the IgG1-WT (A) and IgG1 lysine (B) by weak cationic exchange (WCX). The enzymatic cleaving of lysine by means of carboxypeptidase B shows a reduction in the basic peaks 1 and 2 in the WT IgG1. The overlay (C) of IgG1 WT without CpB (top line) and IgG1 WT+CpB (bottom line) shows the reduction in the basic peak areas by at total of 9.8%. The overlay (D) of IgG1-Lys without CpB (top line) and IgG1-Lys+CpB (bottom line) shows no reduction in the basic peak area (˜below 1%).

FIG. 12: QUANTIFICATION OF THE C-TERMINAL LYSINE BY LC-MS

Quantification of the proportion of C-terminal lysine in the heavy antibody chain of IgG4 by LC-MS (IgG4 WT: dotted (top) line and IgG4-Lys: continuous (bottom) line). After reduction and chromatographic separation, the quantitative amount of the heavy chain with C-terminal lysine (HC 1-447 with lysine) or without lysine (HC 1-446 without lysine) was determined. The arrows indicate the mass shift caused by lysine cleavage dependent on the glycosylation state. Marked peaks characterise glycosylations of the heavy chain (black: HC with C-terminal lysine, grey background: HC without C-terminal lysine).

### DETAILED DESCRIPTION OF THE INVENTION

Definitions

Terms and designations used within the scope of this description of the invention have the following meanings defined hereinafter. The general terms “containing” or “contains” includes the more specific term “consisting of”. Moreover, the terms “single number” and “plurality” are not used restrictively.

The term “titre” is a statement of the product concentration in a defined volume, e.g. ng/mL, mg/mL, mg/L, g/L.

The term “specific productivity” refers to the amount of protein produced by the cell, in pg per cell and per day. It is calculated using the formula pg/((Ct−Co)t/In(Ct−Co)), where Co and Ct indicate the number of cells on seeding or harvesting and t is the cultivation period.

The term “yield” describes the percentage recovery of the various product variants after separation by chromatography on a matrix, e.g. a protein A matrix.

Product concentration of proteins coded by a selected nucleotide sequence may be determined using an ELISA, but also by other methods, such as e.g. protein A HPLC, Western Blot, radioimmunoassay, immunoprecipitation, detection of the biological activity of the protein, immune staining of the protein followed by FACS analysis or fluorescence microscopy, direct detection of a fluorescent protein by FACS analysis or by spectrophotometry.

Gene of Interest:

The gene of interest contained in the expression vector according to the invention comprises a nucleotide sequence of any length which codes for a product of interest. The gene product or “product of interest” is generally a protein, polypeptide, peptide or fragment or derivative thereof. However, it may also be RNA or antisense RNA. The gene of interest may be present in its full length, in shortened form, as a fusion gene or as a labelled gene. It may be genomic DNA or preferably cDNA or corresponding fragments or fusions. The gene of interest may be the native gene sequence, or it may be mutated or otherwise modified. Such modifications include codon optimisations for adapting to a particular host cell and humanisation. The gene of interest may, for example, code for a secreted, cytoplasmic, nuclear-located, membrane-bound or cell surface-bound polypeptide.

The term “nucleic acid”, “nucleotide sequence” or “nucleic acid sequence” indicates an oligonucleotide, nucleotides, polynucleotides and fragments thereof as well as DNA or RNA of genomic or synthetic origin which occur as single or double strands and can represent the coding or non-coding strand of a gene. Nucleic acid sequences may be modified using standard techniques such as site-specific mutagenesis or PCR-mediated mutagenesis.

By “coding” is meant the property or capacity of a specific sequence of nucleotides in a nucleic acid, for example a gene in a chromosome or an mRNA, to act as a matrix for the synthesis of other polymers and macromolecules such as for example rRNA, tRNA, mRNA, other RNA molecules, cDNA or polypeptides in a biological process. Accordingly, a gene codes for a protein if the desired protein is produced in a cell or another biological system by transcription and subsequent translation of the mRNA. Both the coding strand whose nucleotide sequence is identical to the mRNA sequence and is normally also given in sequence databanks, e.g. EMBL or GenBank, and also the non-coding strand of a gene or cDNA which acts as the matrix for transcription may be referred to as coding for a product or protein. A nucleic acid which codes for a protein also includes nucleic acids which have a different order of nucleotide sequence on the basis of the degenerate genetic code but result in the same amino acid sequence of the protein. Nucleic acid sequences which code for proteins may also contain introns.

The term “cDNA” denotes deoxyribonucleic acids which are prepared by reverse transcription and synthesis of the second DNA strand from a mRNA or other RNA produced from a gene. If the cDNA is present as a double stranded DNA molecule it contains both a coding and a non-coding strand.

Protein/Product of Interest

Proteins/polypeptides with a biopharmaceutical significance include for example antibodies or immunoglobulins, enzymes, cytokines, lymphokines, adhesion molecules, receptors and the derivatives or fragments thereof, but are not restricted thereto. Generally, all polypeptides which act as agonists or antagonists and/or have therapeutic or diagnostic applications may be used. Other proteins of interest are, for example, proteins/polypeptides, which are used to change the properties of host cells within the scope of so-called “Cell Engineering”, such as e.g. anti-apoptotic proteins, chaperones, metabolic enzymes, glycosylation enzymes and the derivatives or fragments thereof, but are not restricted thereto.

The term “polypeptides” is used for amino acid sequences or proteins and refers to polymers of amino acids of any length. This term also includes proteins which have been modified post-translationally by reactions such as glycosylation, phosphorylation, acetylation or protein processing. The structure of the polypeptide may be modified, for example, by substitutions, deletions or insertions of amino acids and fusion with other proteins while retaining its biological activity. In addition, the polypeptides may multimerise and form homo- and heteromers.

“Immunoglobulins”, or “antibodies” are proteins selected from among the globulins, which are formed as a reaction of the host organism to a foreign substance (=antigen) from differentiated B-lymphocytes (plasma cells). They serve to defend specifically against these foreign substances. There are various classes of immunoglobulins: IgA, IgD, IgE, IgG, IgM, IgY, IgW. The terms immunoglobulin and antibody are used interchangeably.

Examples of therapeutic antibodies are monoclonal, polyclonal, multispecific and single chain antibodies or immunoglobulins and fragments thereof such as for example Fab, Fab′, F(ab′)₂, Fc and Fc′ fragments, light (L) and heavy (H) immunoglobulin chains and the constant, variable or hypervariable regions thereof as well as Fv and Fd fragments. The antibodies may be of human or non-human origin. Humanised and chimeric antibodies are also possible.

Fab fragments (fragment antigen binding=Fab) consist of the variable regions of both chains which are held together by the adjacent constant regions. They may be produced for example from conventional antibodies by treating with a protease such as papain or by DNA cloning. Other antibody fragments are F(ab′)₂ fragments which can be produced by proteolytic digestion with pepsin.

By gene cloning it is also possible to prepare shortened antibody fragments which consist only of the variable regions of the heavy (VH) and light chain (VL). These are known as Fv fragments (fragment variable=fragment of the variable part). As covalent binding via the cysteine groups of the constant chains is not possible in these Fv fragments, they are often stabilised by some other method. For this purpose the variable regions of the heavy and light chains are often joined together by means of a short peptide fragment of about 10 to 30 amino acids, preferably 15 amino acids. This produces a single polypeptide chain in which VH and VL are joined together by a peptide linker. Such antibody fragments are also referred to as single chain Fv fragments (scFv). Examples of scFv antibodies are known and described.

In past years various strategies have been developed for producing multimeric scFv derivatives. The intention is to produce recombinant antibodies with improved pharmacokinetic properties and increased binding avidity. In order to achieve the multimerisation of the scFv fragments they are produced as fusion proteins with multimerisation domains. The multimerisation domains may be, for example, the CH3 region of an IgG or helix structures (“coiled coil structures”) such as the Leucine Zipper domains. In other strategies the interactions between the VH and VL regions of the scFv fragment are used for multimerisation (e.g. dia-, tri- and pentabodies).

The term “diabody” is used in the art to denote a bivalent homodimeric scFv derivative. Shortening the peptide linker in the scFv molecule to 5 to 10 amino acids results in the formation of homodimers by superimposing VH/VL chains. The diabodies may additionally be stabilised by inserted disulphide bridges. Examples of diabodies can be found in the literature.

The term “minibody” is used in the art to denote a bivalent homodimeric scFv derivative. It consists of a fusion protein which contains the CH3 region of an immunoglobulin, preferably IgG, most preferably IgG1, as dimerisation region. This connects the scFv fragments by means of a hinge region, also of IgG, and a linker region.

The term “triabody” is used in the art to denote a trivalent homotrimeric scFv derivative. The direct fusion of VH-VL without the use of a linker sequence leads to the formation of trimers.

The fragments known in the art as mini antibodies which have a bi-, tri- or tetravalent structure are also derivatives of scFv fragments. The multimerisation is achieved by means of di-, tri- or tetrameric coiled coil structures.

Preparation of Expression Vectors According to the Invention:

The expression vector according to the invention may theoretically be prepared by conventional methods known in the art. There is also a description of the functional components of a vector, e.g. suitable promoters, enhancers, termination and polyadenylation signals, antibiotic resistance genes, selectable markers, replication starting points and splicing signals. Conventional cloning vectors may be used to produce them, e.g. plasmids, bacteriophages, phagemids, cosmids or viral vectors such as baculovirus, retroviruses, adenoviruses, adeno-associated viruses and herpes simplex virus, as well as synthetic or artificial chromosomes or mini-chromosomes. The eukaryotic expression vectors typically also contain prokaryotic sequences such as, for example, replication origin and antibiotic resistance genes which allow replication and selection of the vector in bacteria. A number of eukaryotic expression vectors which contain multiple cloning sites for the introduction of a polynucleotide sequence are known and some may be obtained commercially from various companies such as Stratagene, La Jolla, Calif., USA; Invitrogen, Carlsbad, Calif., USA; Promega, Madison, Wis., USA or BD Biosciences Clontech, Palo Alto, Calif., USA.

Fundamentally, the expression of the genes within an expression vector may take place starting from one or more transcription units. The term transcription unit is defined as a region which contains one or more genes to be transcribed. The genes within a transcription unit are functionally linked to one another in such a way that all the genes within such a unit are under the transcriptional control of the same promoter or promoter/enhancer. As a result of this transcriptional linking of genes, more than one protein or product can be transcribed from a transcription unit and thus expressed. Each transcription unit contains the regulatory elements which are necessary for the transcription and translation of the gene sequences contained therein. Each transcription unit may contain the same or different regulatory elements. IRES elements or introns may be used for the functional linking of the genes within a transcription unit.

The expression vector may contain a single transcription unit for expressing the gene (or genes) of interest and selectable marker genes, for example. Alternatively, these genes may also be arranged in two or more transcription units. Various combinations of the genes within a transcription unit are possible. In another embodiment of the present invention more than one expression vector consisting of one, two or more transcription units may be inserted in a host cell by cotransfection or in successive transfections in any desired order. Any combination of regulatory elements and genes on each vector can be selected provided that adequate expression of the transcription units is ensured. If necessary, other regulatory elements and genes, e.g. additional genes of interest or selectable markers, may be positioned on the expression vectors.

Host Cells:

For transfection with the expression vector according to the invention eukaryotic host cells are used, preferably mammalian cells and more particularly rodent cells such as mouse, rat and hamster cell lines. The successful transfection of the corresponding cells with an expression vector according to the invention results in transformed, genetically modified, recombinant or transgenic cells, which are also the subject of the present invention.

Preferred host cells for the purposes of the invention are hamster cells such as BHK21, BHK TK⁻, CHO, CHO-K1, CHO-DUKX, CHO-DUKX B1 and CHO-DG44 cells or derivatives/descendants of these cell lines. Particularly preferred are CHO-DG44, CHO-DUKX, CHO-K1 and BHK21 cells, particularly CHO-DG44 and CHO-DUKX cells. Also suitable are myeloma cells from the mouse, preferably NS0 and Sp2/0 cells and derivatives/descendants of these cell lines. However, derivatives and descendants of these cells, other mammalian cells including but not restricted to cell lines of humans, mice, rats, monkeys, rodents, or eukaryotic cells, including but not restricted to yeast, insect, bird and plant cells, may also be used as host cells for the production of biopharmaceutical proteins.

The transfection of the eukaryotic host cells with a polynucleotide or one of the expression vectors according to the invention is carried out by conventional methods. Suitable methods of transfection include for example liposome-mediated transfection, calcium phosphate coprecipitation, electroporation, polycation- (e.g. DEAE dextran)-mediated transfection, protoplast fusion, microinjection and viral infections. According to the invention stable transfection is preferably carried out in which the constructs are either integrated into the genome of the host cell or an artificial chromosome/minichromosome, or are episomally contained in stable manner in the host cell. The transfection method which gives the optimum transfection frequency and expression of the heterologous gene in the host cell in question is preferred. By definition, every sequence or every gene inserted in a host cell is referred to as a “heterologous sequence” or “heterologous gene” in relation to the host cell. This applies even if the sequence to be introduced or the gene to be introduced is identical to an endogenous sequence or an endogenous gene of the host cell. For example, a hamster actin gene introduced into a hamster host cell is by definition a heterologous gene.

In the recombinant production of heteromeric proteins such as e.g. monoclonal antibodies (mAb), the transfection of suitable host cells can theoretically be carried out by two different methods. mAb's of this kind are composed of a number of subunits, the heavy and light chains. Genes coding for these subunits may be accommodated in independent or multicistronic transcription units on a single plasmid with which the host cell is then transfected. This is intended to secure the stoichiometric representation of the genes after integration into the genome of the host cell. However, in the case of independent transcription units it must hereby be ensured that the mRNAs which encode the different proteins display the same stability and transcriptional and translational efficiency. In the second case, the expression of the genes take place within a multicistronic transcription unit by means of a single promoter and only one transcript is formed.

By using IRES elements, a highly efficient internal translation initiation of the genes is obtained in the second and subsequent cistrons. However, the expression rates for these cistrons are lower than that of the first cistron, the translation initiation of which, by means of a so-called “cap”-dependent pre-initiation complex, is substantially more efficient than IRES-dependent translation initiation. In order to achieve a truly equimolar expression of the cistrons, additional inter-cistronic elements may be introduced, for example, which ensure uniform expression rates in conjunction with the IRES elements.

Another possible way of simultaneously producing a number of heterologous proteins, which is preferred according to the invention, is cotransfection, in which the genes are separately integrated in different expression vectors. This has the advantage that certain proportions of genes and gene products to one another can be selected, thereby balancing out any differences in the mRNA stability and in the efficiency of transcription and translation. In addition, the expression vectors are more stable because of their small size and are easier to handle both during cloning and during transfection.

In one particular embodiment of the invention, therefore, the host cells are additionally transfected, preferably cotransfected, with one or more vectors having genes which code for one or more other proteins of interest. The other vector or vectors used for the cotransfection code, for example, for the other protein or proteins of interest under the control of the same promoter, preferably under the control of the same promoter/enhancer combination, and for at least one selectable marker, e.g. dihydrofolate reductase.

In another particular embodiment of the invention the host cells are co-transfected with at least two eukaryotic expression vectors, at least one of the two vectors containing at least one gene which codes for at least the protein of interest, while the other vector contains one or more nucleic acids according to the invention in any combination, position and orientation, and optionally also codes for at least one gene of interest, and these nucleic acids according to the invention impart their transcription- or expression-enhancing activity to the genes of interest which are located on the other co-transfected vector, by co-integration with the other vector.

According to the invention the host cells are preferably established, adapted and cultivated under serum-free conditions, optionally in media which are free from animal proteins/peptides. Examples of commercially obtainable media include Ham's F12 (Sigma, Deisenhofen, Del.), RPMI-1640 (Sigma), Dulbecco's Modified Eagle's Medium (DMEM; Sigma), Minimal Essential Medium (MEM; Sigma), Iscove's Modified Dulbecco's Medium (IMDM; Sigma), CD-CHO (Invitrogen, Carlsbad, Calif., USA), CHO-S-SFMII (Invitrogen), serum-free CHO-Medium (Sigma) and protein-free CHO-Medium (Sigma). Each of these media may optionally be supplemented with various compounds, e.g. hormones and/or other growth factors (e.g. insulin, transferrin, epidermal growth factor, insulin-like growth factor), salts (e.g. sodium chloride, calcium, magnesium, phosphate), buffers (e.g. HEPES), nucleosides (e.g. adenosine, thymidine), glutamine, glucose or other equivalent nutrients, antibiotics and/or trace elements. Although serum-free media are preferred according to the invention, the host cells may also be cultivated using media which have been mixed with a suitable amount of serum. In order to select genetically modified cells which express one or more selectable marker genes, one or more selecting agents are added to the medium.

The term “selecting agent” refers to a substance which affects the growth or survival of host cells with a deficiency for the selectable marker gene in question. Within the scope of the present invention, geneticin (G418) is preferably used as the medium additive for the selection of heterologous host cells which carry a wild-type or preferably a modified neomycin phosphotransferase gene. If the host cells are to be transfected with a number of expression vectors, e.g. if several genes of interest are to be separately introduced into the host cell, they generally have different selectable marker genes.

A “selectable marker gene” is a gene which allows the specific selection of cells which contain this gene by the addition of a corresponding selecting agent to the cultivation medium. As an illustration, an antibiotic resistance gene may be used as a positive selectable marker. Only cells which have been transformed with this gene are able to grow in the presence of the corresponding antibiotic and thus be selected. Untransformed cells, on the other hand, are unable to grow or survive under these selection conditions. There are positive, negative and bifunctional selectable markers. Positive selectable markers permit the selection and hence enrichment of transformed cells by conferring resistance to the selecting agent or by compensating for a metabolic or catabolic defect in the host cell. By contrast, cells which have received the gene for the selectable marker can be selectively eliminated by negative selectable markers. An example of this is the thymidine kinase gene of the Herpes Simplex virus, the expression of which in cells with the simultaneous addition of acyclovir or gancyclovir leads to the elimination thereof. The selectable markers used in this invention, including the amplifiable selectable markers, include genetically modified mutants and variants, fragments, functional equivalents, derivatives, homologues and fusions with other proteins or peptides, provided that the selectable marker retains its selective qualities. Such derivatives display considerable homology in the amino acid sequence in the regions or domains which are deemed to be selective. The literature describes a large number of selectable marker genes including bifunctional (positive/negative) markers. Examples of selectable markers which are usually used in eukaryotic cells include the genes for aminoglycoside phosphotransferase (APH), hygromycine phosphostransferase (HYG), dihydrofolate reductase (DHFR), thymidine kinase (TK), glutamine synthetase, asparagin synthetase and genes which confer resistance to neomycin (G418), puromycin, histidinol D, bleomycin, phleomycin and zeocin.

Amplifiable Selectable Marker Gene:

In addition, the cells according to the invention may optionally also be subjected to one or more gene amplification steps in which they are cultivated in the presence of a selecting agent which leads to amplification of an amplifiable selectable marker gene.

The prerequisite is that the host cells are additionally transfected with a gene which codes for an amplifiable selectable marker. It is conceivable for the gene which codes for an amplifiable selectable marker to be present on one of the expression vectors according to the invention or to be introduced into the host cell by means of another vector.

The amplifiable selectable marker gene usually codes for an enzyme which is needed for the growth of eukaryotic cells under certain cultivation conditions. For example, the amplifiable selectable marker gene may code for dihydrofolate reductase (DHFR). In this case the gene is amplified if a host cell transfected therewith is cultivated in the presence of the selecting agent methotrexate (MTX).

The DHFR marker is particularly suitable for the selection and subsequent amplification when using DHFR-negative basic cells such as CHO-DG44 or CHO-DUKX, as these cells do not express endogenous DHFR and therefore do not grow in purine-free medium. Consequently, the DHFR gene may be used here as a dominant selectable marker and the transformed cells are selected in hypoxanthine/thymidine-free medium.

Other amplifiable selectable marker genes which may be used according to the invention are for example glutamine-synthetase, methallothioneine, adenosine-deaminase, AMP-deaminase, UMP-synthase, xanthine-guanine-phosphoribosyltransferase and thymdilate-synthetase.

Gene Expression and Selection of High-Producing Host Cells:

The term “gene expression” or “expression” relates to the transcription and/or translation of a heterologous gene sequence in a host cell. The expression rate can be generally determined, either on the basis of the quantity of corresponding mRNA which is present in the host cell or on the basis of the quantity of gene product produced which is encoded by the gene of interest. The quantity of mRNA produced by transcription of a selected nucleotide sequence can be determined for example by northern blot hybridisation, ribonuclease-RNA-protection, in situ hybridisation of cellular RNA or by PCR methods (e.g. quantitative PCR). Proteins which are encoded by a selected nucleotide sequence can also be determined by various methods such as, for example, ELISA, protein A HPLC, western blot, radioimmunoassay, immunoprecipitation, detection of the biological activity of the protein, immune staining of the protein followed by FACS analysis or fluorescence microscopy, direct detection of a fluorescent protein by FACS analysis or fluorescence microscopy.

By “increased titre or productivity” is meant the increase in expression, synthesis or secretion of a heterologous sequence introduced into a host cell, for example of a gene coding for a therapeutic protein, by comparison with a suitable control, for example mutant protein versus wild-type protein. There is increased titre or productivity if a cell according to the invention is cultivated according to a method according to the invention described here, and if this cell has at least a 10% increase in specific productivity or titre. There is also increased titre or productivity if a cell according to the invention is cultivated according to a method according to the invention described here, and if this cell has at least a 20% or at least 50% or at least 75% increase in specific productivity or titre. There is also in particular increased titre or productivity if a cell according to the invention is cultivated according to a method according to the invention described here, and if this cell has at least a 10-500%, preferably 20-300%, particularly preferably 50-200% increase in specific productivity or titre.

An increased titre or productivity may be obtained both by using one of the expression vectors according to the invention and also by using one of the processes according to the invention.

The corresponding processes may be combined with a FACS-assisted selection of recombinant host cells which contain, as additional selectable marker, one or more fluorescent proteins (e.g. GFP) or a cell surface marker. Other methods of obtaining increased expression, and a combination of different methods may also be used, are based for example on the use of cis-active elements for manipulating the chromatin structure (e.g. LCR, UCOE, EASE, isolators, S/MARs, STAR elements), on the use of (artificial) transcription factors, treatment of the cells with natural or synthetic agents for up-regulating endogenous or heterologous gene expression, improving the stability (half-life) of mRNA or the protein, improving the initiation of mRNA translation, increasing the gene dose by the use of episomal plasmids (based on the use of viral sequences as replication origins, e.g. SV40, polyoma, adenovirus, EBV or BPV), the use of amplification-promoting sequences or in vitro amplification systems based on DNA concatemers.

Also preferred according to the invention is a process in which production cells are replicated and used to prepare the coding gene product of interest. For this, the selected high producing cells are preferably cultivated in a serum-free culture medium and preferably in suspension culture under conditions which allow expression of the gene of interest. The protein/product of interest is preferably obtained from the cell culture medium as a secreted gene product. If the protein is expressed without a secretion signal, however, the gene product may also be isolated from cell lysates. In order to obtain a pure homogeneous product which is substantially free from other recombinant proteins and host cell proteins, conventional purification procedures are carried out. First of all, cells and cell debris are removed from the culture medium or lysate. The desired gene product can then be freed from contaminating soluble proteins, polypeptides and nucleic acids, e.g. by fractionation on immunoaffinity and ion exchange columns, ethanol precipitation, reversed phase HPLC or chromatography on Sephadex, silica or cation exchange resins such as DEAE. Methods which result in the purification of a heterologous protein expressed by recombinant host cells are known to the skilled man and described in the literature.

### Embodiments According to the Invention

The present invention relates to a method for increasing the titre of a protein of interest of a cell, characterised in that
a. in a nucleic acid sequence which codes for the protein of interest, at least the codon which codes for the C-terminal amino acid is deleted,
b. the cell is transfected with a vector, which contains the modified nucleic acid from a) and
c. the cell is cultivated under conditions that permit the production of the protein of interest.

In particular the present invention relates to a method for increasing the titre of an antibody of a cell characterised in that in a nucleic acid sequence, which codes for the heavy chain of the antibody, at least the codon which codes for the C-terminal amino acid lysine is deleted, the cell is transfected with a vector which contains the modified nucleic acid, and the cell is cultivated under conditions that allow production of the antibody of interest.

The present invention preferably relates to a method for increasing the specific productivity of a protein of interest of a cell, characterised in that in a nucleic acid sequence which codes for the protein of interest, at least the codon which codes for the C-terminal amino acid is deleted, the cell is transfected with a vector, which contains the modified nucleic acid, and the cell is cultivated under conditions that permit the production of the protein of interest.

In a particularly preferred embodiment the present invention relates to a method for increasing the specific productivity of an antibody of a cell, characterised in that in a nucleic acid sequence which codes for the heavy chain of the antibody, at least the codon which codes for the C-terminal amino acid lysine is deleted, the cell is transfected with a first vector, which contains the modified nucleic acid, the cell is co-transfected with a second vector, which contains the light chain of an antibody, and the cell is cultivated under conditions that permit production of the antibody.

In another preferred method the modified heavy chain and the light chain, or the subunits of a heteromeric protein, are incorporated in successive transfections in any desired order.

In another preferred embodiment the present invention relates to a method for increasing the specific productivity of an antibody or of any heteromeric protein of interest of a cell, characterised in that in a nucleic acid sequence which codes for the heavy chain of the antibody, at least the codon which codes for the C-terminal amino acid lysine is deleted, the cell is transfected with a vector which contains both the modified nucleic acid for the heavy chain of an antibody as also the light chain of an antibody, and the cell is cultivated under conditions that permit production of the antibody. In a preferred embodiment of the method the vector with which the cell is transfected is a bi- or multicistronic vector. In another preferred embodiment of the method the vector with which the cell is transfected is a vector which contains the heavy and light antibody chain as separate transcription units.

It may surprisingly be shown that e.g. an IgG1 molecule is expressed and secreted in CHO-cells in spite of the deletion of the C-terminal lysine and the amount of product is comparable with that of IgG1 wild-type transfected cells (FIG. 2). It has also surprisingly been shown that cells that express the lysine deletion variant of IgG1 on average achieve even 27% higher titres or 32% higher specific productivities than cells which express the IgG1 wild-type (FIG. 3). This production advantage of the lysine deletion variant is still present even when a DHFR-based gene amplification is induced in these cell pools by the addition of 100 nM MTX. The titres and specific productivities are on average 86% or 120% higher (FIG. 4).

Similar results may surprisingly also be shown for an IgG4 molecule. In spite of the deletion of the C-terminal lysine the IgG4 molecule is expressed and secreted in CHO-cells even rather better than the IgG4 wild-type (FIG. 5). Surprisingly it is found that cells which express the lysine deletion variant of IgG4, on average even achieve a 63% higher titre or 70% higher specific productivities than cells which express the IgG4-wild-type (FIG. 6). This production advantage of the lysine deletion variant is also present in the following amplification step with 400 nM MTX. On average 53% higher titres and 66% higher specific productivities are obtained (FIG. 7).

In a special embodiment of the method according to the invention the titre and/or the specific productivity is increased by 10-500%, preferably 20-300%, particularly preferably 50-200% based on the comparison value of the protein without the deletion of the C-terminal amino acid. In another special embodiment of the method according to the invention, the titre and/or the specific productivity is increased by at least 10%, preferably by at least 20%, more preferably by at least 50%, and particularly preferably by at least 75% based on the comparison value of the protein without the deletion of the C-terminal amino acid.

In another special embodiment of the method according to the invention the specific productivity is at least 5 pg/cell/day.

The present invention also relates to a method for producing an expression vector for the increased production of a protein of interest characterised in that in the nucleic acid sequence which codes for the protein of interest, at least the codon which codes for the C-terminal amino acid is deleted, and the nucleic acid sequence thus modified is inserted in an expression vector.

The present invention also relates to a method for producing a cell with an increased titre and/or increased specific productivity of a protein of interest, characterised in that a cell is treated using a method according to the invention and then a single cell cloning is carried out, for example by dilution cloning or FACS-based single cell deposition.

The present invention also relates to a process for preparing a protein of interest in a cell, characterised in that a group of cells is treated using a method according to the invention, these cells are selected in the presence of at least one selection pressure, a single cell cloning is optionally carried out and the protein of interest is obtained from the cells or the culture supernatant.

A special embodiment of the method according to the invention for preparing at least one protein of interest is characterised in that the cells used for the preparation, after the selection step using a selection agent, are additionally subjected to a gene amplification step.

A specific embodiment of all the methods described according to the invention is characterised in that the C-terminal amino acid lysine is (Lys) or arginine (Arg), preferably Lys.

Another specific embodiment of all the methods described according to the invention is characterised in that the protein of interest is an antibody, an Fc fusion protein, EPO or tPA.

A preferred embodiment of all the methods described according to the invention is characterised in that the protein of interest is a heavy chain of an antibody and the C-terminal amino acid is lysine (Lys).

A special embodiment of all the methods described according to the invention is characterised in that the heavy chain of the antibody is of the type IgG1, IgG2, IgG3 or IgG4, preferably type IgG1, IgG4 or IgG2.

A specific embodiment of all the methods described according to the invention is characterised in that the protein of interest is a monoclonal, polyclonal, mammalian, murine, chimeric, humanised, primate or human antibody or an antibody fragment or derivative of a heavy chain of an immunoglobulin antibody or of a Fab, F(ab′)2, Fc, Fc-Fc fusion protein, Fv, single chain Fv, single domain Fv, tetravalent single chain Fv, disulphide-linked Fv, domain-deleted antibody, a minibody, diabody or a fusion polypeptide of one of the above-mentioned fragments with another peptide or polypeptide or an Fc-peptide fusion protein, an Fc-toxin fusion protein or a scaffold protein.

Another specific embodiment of all the methods described according to the invention is characterised in that the cell is cultivated in suspension culture. A particular embodiment of all the methods described according to the invention is characterised in that the cell is cultivated under serum-free conditions. Another particular embodiment of all the methods described according to the invention is characterised in that the cell is cultivated in chemically defined medium. A preferred embodiment of all the methods described according to the invention is characterised in that the cell is cultivated in protein-free medium.

A preferred embodiment of all the methods described according to the invention is characterised in that the cell is a eukaryotic cell, e.g. from yeast, plants, worms, insects, birds, fish, reptiles or mammals. Another preferred embodiment of all the methods described according to the invention is characterised in that the cell is a mammalian cell. A particularly preferred embodiment of all the methods described according to the invention is characterised in that the cell is a CHO cell. Another special embodiment of the methods mentioned according to the invention is characterised in that the CHO cell is selected from the group: CHO wild type, CHO K1, CHO DG44, CHO DUKX-B11 and CHO per-5. Particularly preferably it is a CHO DG44 cell.

The invention also relates to an expression vector with increased expression of a gene of interest which may be generated according to one of the methods mentioned according to the invention.

The invention also relates to a cell which may be generated according to one of the methods mentioned according to the invention.

The invention also relates to a method for the production and purification of a protein of interest, characterised in that at least one C-terminal amino acid of the corresponding gene of interest is deleted and the resulting protein of interest has decreased heterogeneity compared with the wild-type protein without deletion.

A particular embodiment of the method according to the invention is characterised in that the C-terminal amino acid is lysine (Lys) or arginine (Arg), preferably Lys.

Another particular embodiment of the method according to the invention is characterised in that the protein of interest is an antibody, an Fc fusion protein, EPO or tPA.

A preferred embodiment of the method according to the invention is characterised in that the protein of interest is a heavy chain of an antibody and the C-terminal amino acid is lysine (Lys).

Another preferred embodiment of the method according to the invention is characterised in that the heavy chain of the antibody is of the type IgG1, IgG2, IgG3 or IgG4, preferably of type IgG1, IgG2 or IgG4.

A particular embodiment of the method according to the invention is characterised in that, for the production, cells are cultivated in suspension culture. Another particular embodiment of the method according to the invention is characterised in that for the production cells are cultivated under serum-free conditions. Another particular embodiment of all the methods described according to the invention is characterised in that the cell is cultivated in chemically defined medium. A preferred embodiment of all the methods described according to the invention is characterised in that the cell is cultivated in protein-free medium.

A preferred embodiment of the method according to the invention is characterised in that the cells are mammalian cells.

Another preferred embodiment of the method according to the invention is characterised in that the cells are CHO cells, preferably CHO DG44 cells.

A particularly preferred embodiment of the method according to the invention is characterised in that during the purification of the protein of interest a lower salt concentration is used compared with the purification of a wild-type protein without deletion.

The invention also relates to a process for preparing an antibody characterised in that in a nucleic acid which codes for the heavy chain of an antibody, at least the codon which codes for the C-terminal amino acid lysine is deleted, the cell is transfected with a vector, which contains the nucleic acid thus modified, and the cell is cultivated under conditions that allow expression of the antibody.

The invention is hereinafter explained more fully by means of non-restrictive embodiments by way of example.

### Examples

Abbreviations

AP: alkaline phosphatase

Asp (=D): aspartic acid

bp: base pair

CHO: Chinese Hamster Ovary

CpB: carboxypeptidase B

DHFR: dihydrofolate-reductase

ELISA: enzyme-linked immunosorbant assay

HT: hypoxanthine/thymidine

IgG: immunoglobulin G

Ile (=I): isoleucine

kb: kilobase

Lys: lysine

mAk: monoclonal antibody

MTX: methotrexate

MW: mean value

NPT: neomycin-phosphotransferase

PCR: polymerase chain reaction

phe (=F): phenylalanine

SEAP: secreted alkaline phosphatase

WT: wild-type

Methods

Cell Culture and Transfection

The cells CHO-DG44/dhfr^{-/-} are permanent cultivated as suspension cells in serum-free CHO-S-SFMII medium supplemented with hypoxanthine and thymidine (HT) (Invitrogen GmbH, Karlsruhe, Del.) in cell culture flasks at 37° C. in a damp atmosphere and 5% CO₂. The cell counts and viability are determined with a Cedex (Innovatis) and the cells are then seeded in a concentration of 1-3×10⁵/mL and run every 2-3 days.

For the transfection of CHO-DG44, Lipofectamine Plus Reagent (Invitrogen) is used. For each transfection batch a total of 1.0-1.1 μg plasmid-DNA, 4 μL Lipofectamine and 6 μL Plus reagent are mixed according to the manufacturers' instructions and added in a volume of 200 μL to 6×10⁵ cells in 0.8 ml HT-supplemented CHO-S-SFMII medium. After three hours' incubation at 37° C. in a cell incubator 2 mL of HT-supplemented CHO-S-SFMII medium are added. After a cultivation period of 48 hours the transfection mixtures are either harvested (transient transfection) or subjected to selection. As one expression vector contains a DHFR selection marker and the other one contains an NPT selection marker, 2 days after transfection the co-transfected cells are transferred into CHO-S-SFMII medium without added hypoxanthine and thymidine for the DHFR- and NPT-based selection and G418 (Invitrogen) is also added to the medium in a concentration of 400 μg/mL.

A DHFR-based gene amplification of the integrated heterologous genes is carried out by the addition of the selection agent MTX (Sigma, Deisenhofen, Del.) in a concentration of 5-2000 nM to an HT-free CHO-S-SFMII medium.

Expression Vectors

For the expression analysis eukaryotic expression vectors are used which are based on the pAD-CMV vector and mediate the expression of a heterologous gene via the combination of CMV enhancer/hamster ubiquitin/S27a promoter (WO 97/15664) or CMV enhancer/CMV promoter. Whereas the base vector pBID contains the dhfr minigene which acts as an amplifiable selectable marker, in the vector pBIN the dhfr-minigene is replaced by an NPT gene. For this purpose the NPT selection marker, including SV40 early promoter and TK-polyadenylation signal, was isolated from the commercial plasmid pBK-CMV (Stratagene, La Jolla, Calif., USA) as a 1640 by Bsu36I fragment. After a reaction of topping up the fragment ends with Klenow DNA polymerase the fragment was ligated with the 3750 by Bsu361/StuI fragment of the vector pBID, which was also treated with Klenow DNA polymerase. In both vectors the expression of the heterologous gene is controlled via the combination of CMV enhancer/hamster ubiquitin/S27a promoter.

The vector pBIN8a is a derivative of the vector pBIN and contains a modified NPT gene. It is the NPT variant F240I (Phe240Ile), the cloning of which is described in WO2004/050884. In this vector and also in the vector pBIDa, a derivative of the vector pBID, the expression of the heterologous gene is under the control of the CMV enhancer/promoter combination.

ELISA (Enzyme-Linked Immunosorbant Assay)

The quantification of the expressed antibodies (IgG1, IgG2 or IgG4) in the supernatants of stably transfected CHO-DG44 cells is carried out using ELISA according to standard procedures, using on the one hand a goat anti human IgG Fc fragment (Dianova, Hamburg, Del.) and on the other hand an AP-conjugated goat anti human kappa light chain antibody (Sigma). The standard used is purified antibody of the same isotype as the expressed antibodies in each case.

Productivities (pg/cell/day) are calculated with the formula pg/((Ct−Co)t/In(Ct−Co)), where Co and Ct indicate the cell count on seeding or harvesting and t represents the cultivation period.

SEAP Assay

The SEAP titre in culture supernatants from transiently transfected CHO-DG44 cells is quantified using the SEAP Reporter Gene Assays according to the manufacturer's operating instructions (Roche Diagnostics GmbH, Mannheim, Del.).

### Example 1

### Cloning and Expression of IGG1 with C-Terminal Lysine Deletion

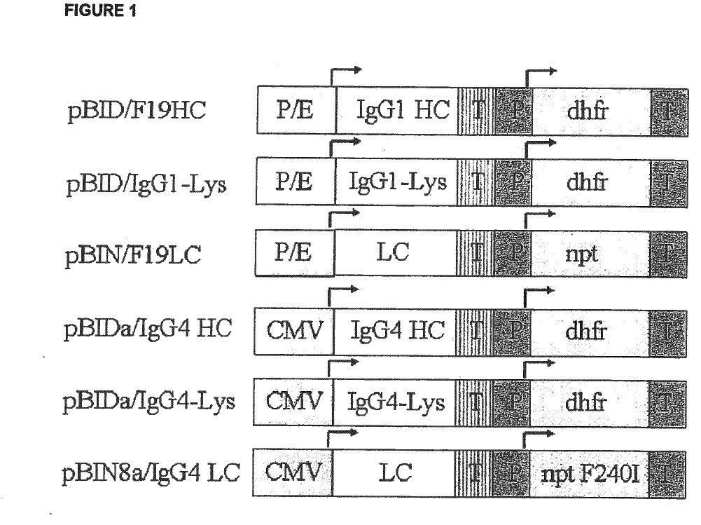

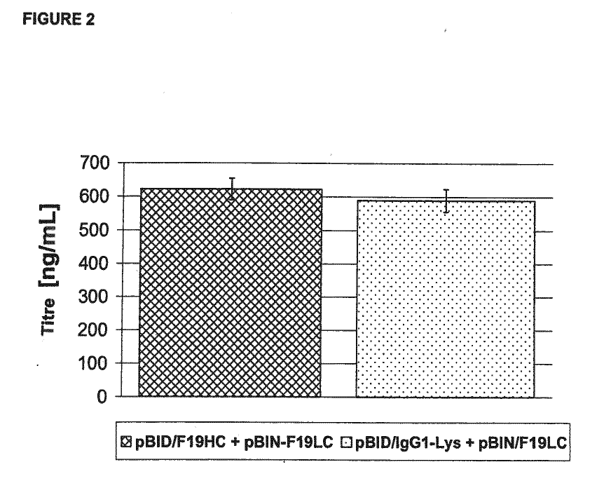

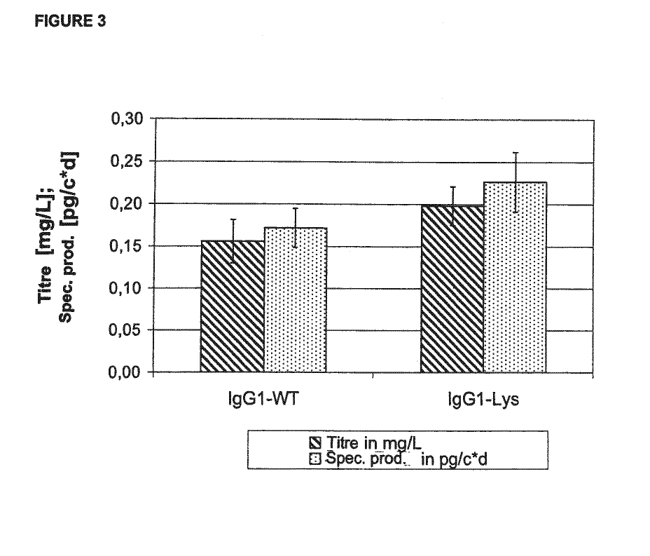

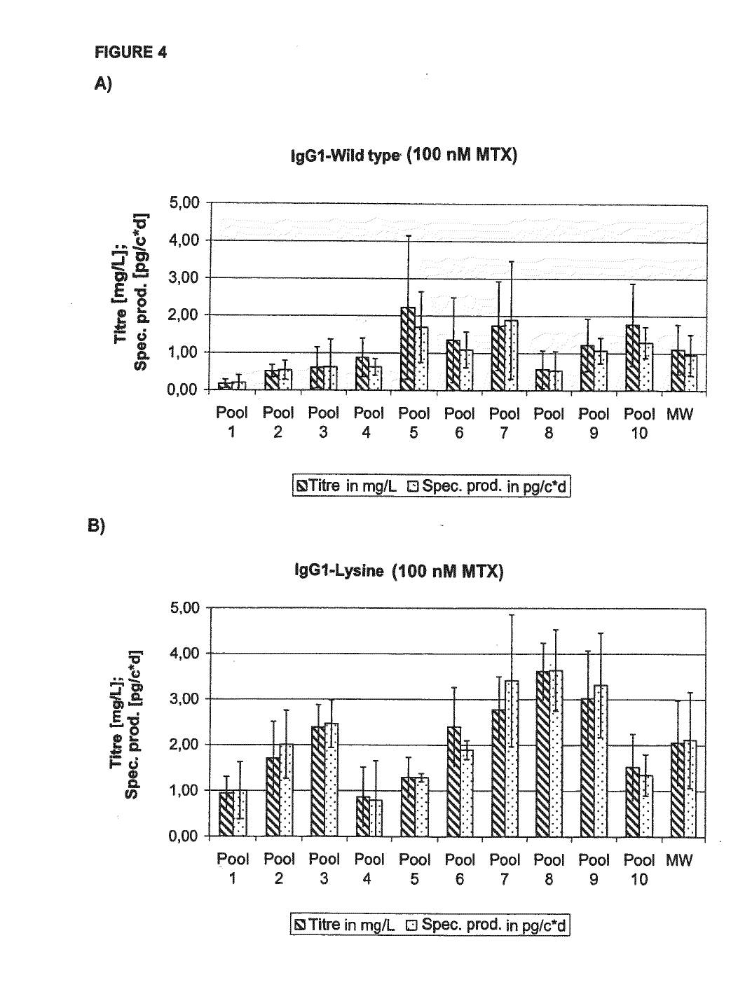

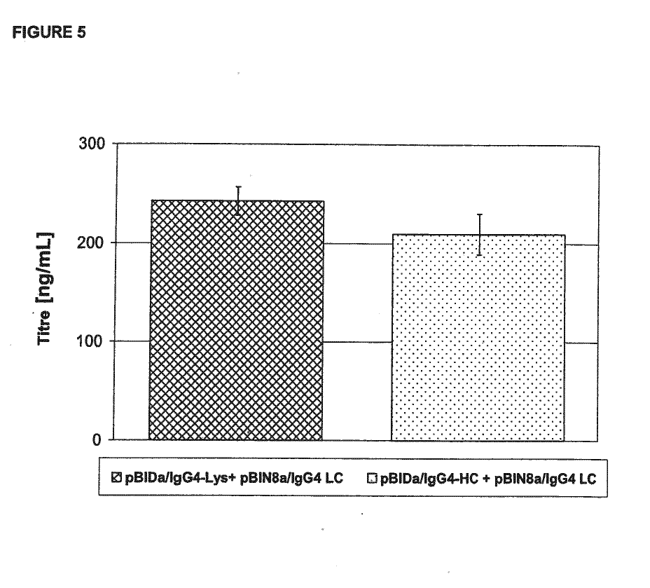

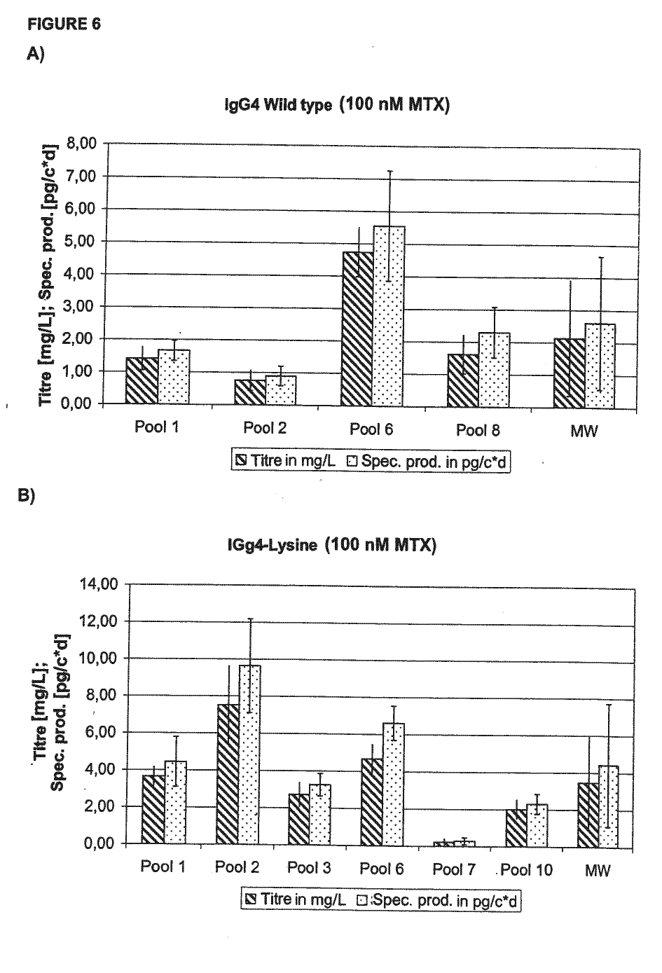

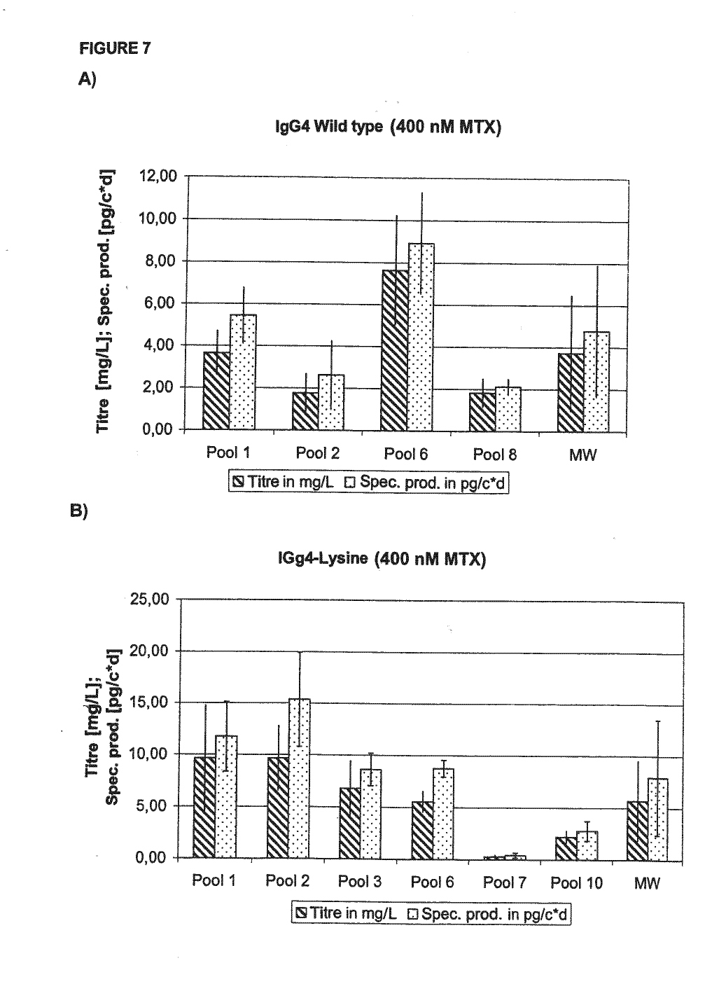

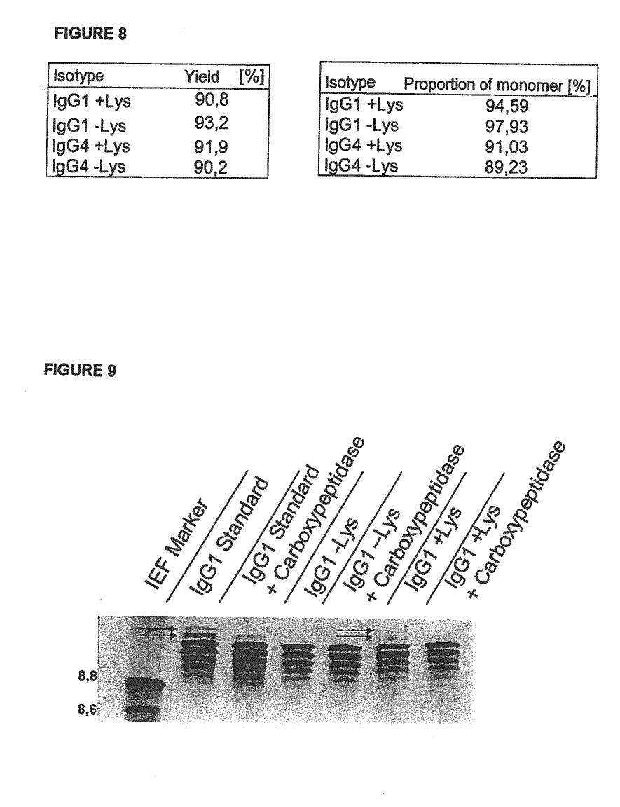

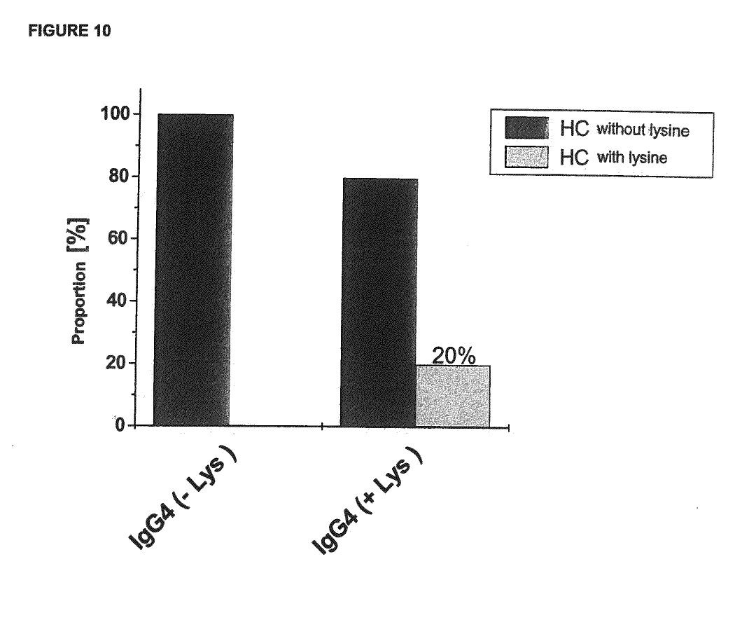

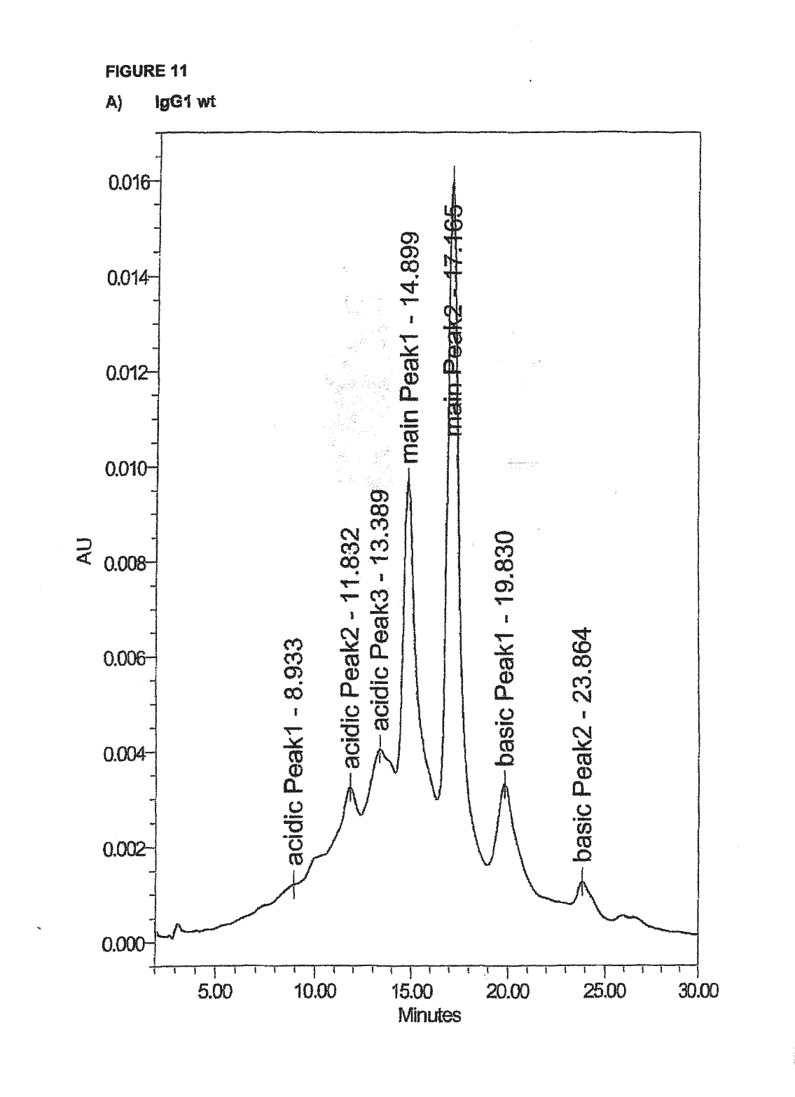

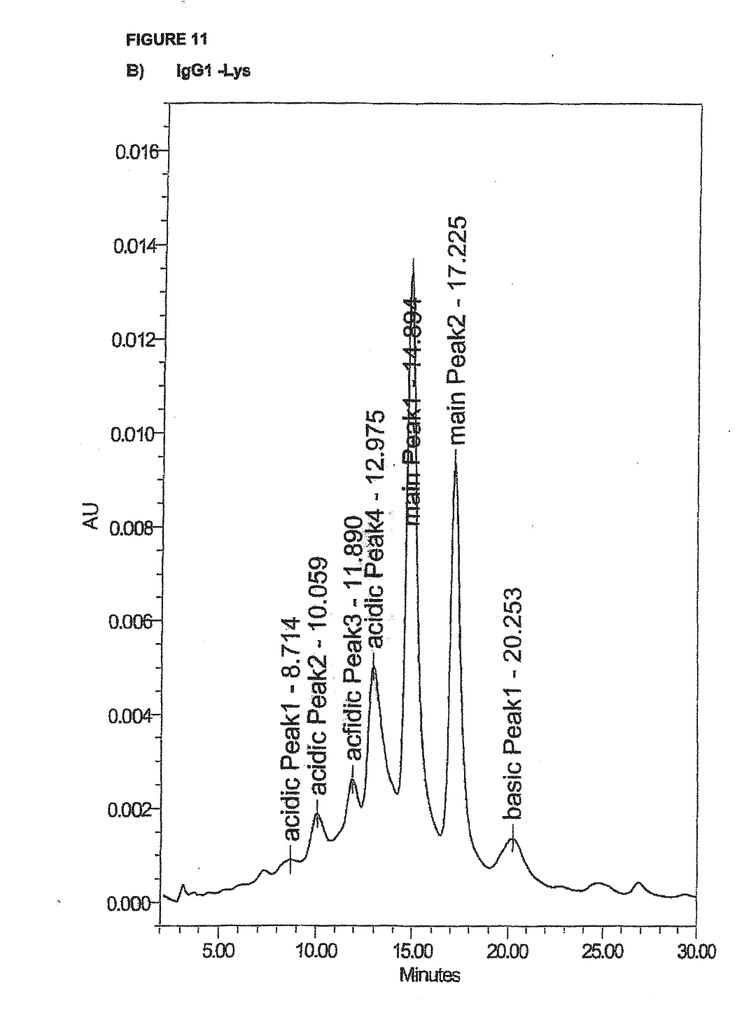

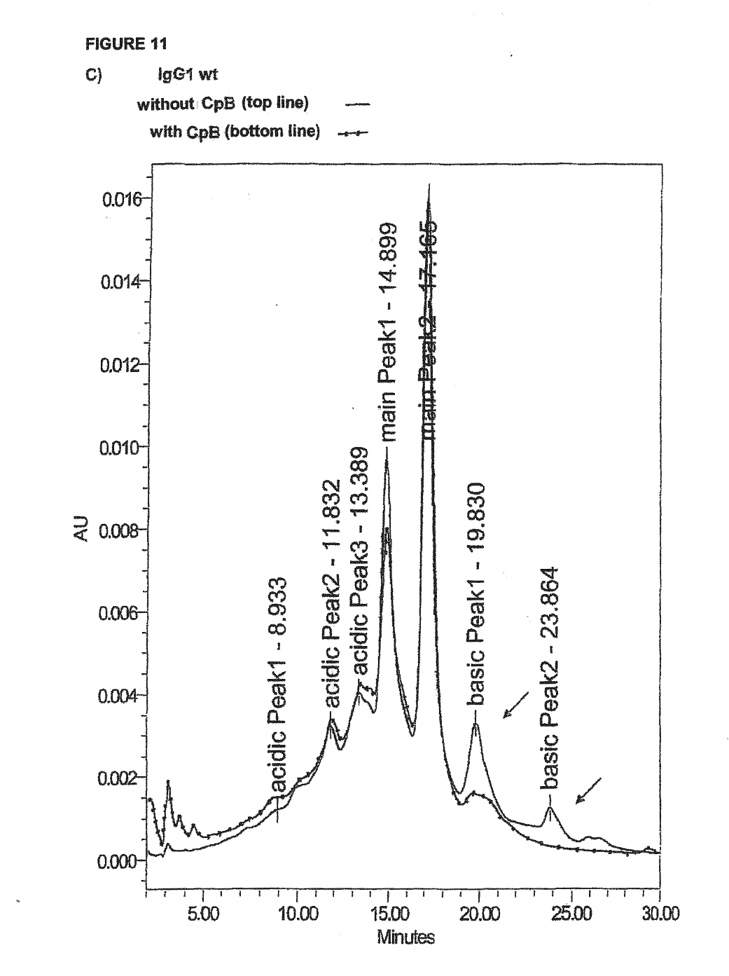

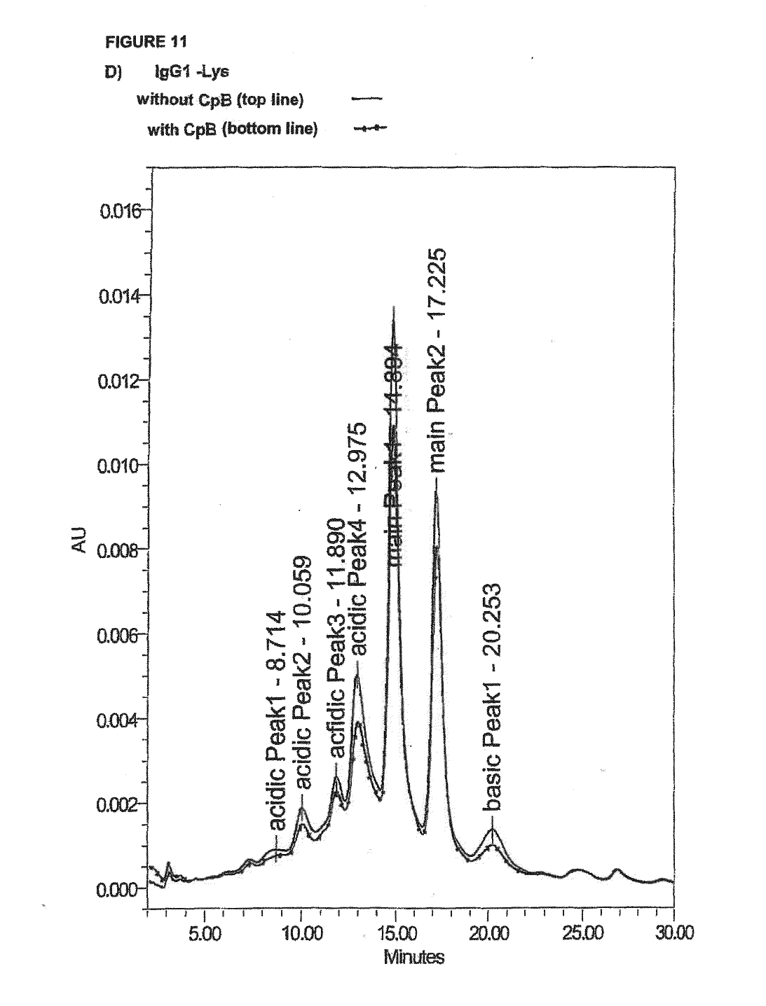

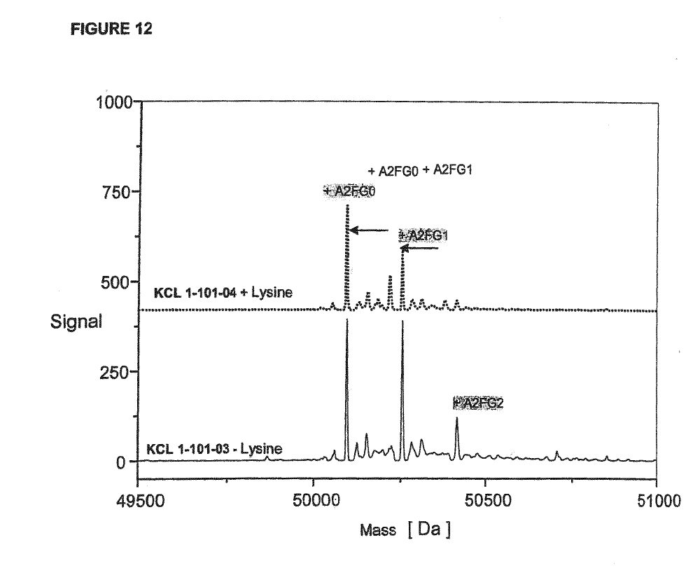

The heavy chain of the monoclonal humanised F19 antibody (IgG1/kappa) is isolated from the plasmid pG1D105F19HC (NAGENESEQ: AAZ32786) as a 1.5 kb NaeI/HindIII fragment and cloned into the vector pBID digested with EcoRI (topped up with Klenow-DNA-polymerase) and HindIII, to produce the vector pBID/F19HC (FIG. 1). The light chain on the other hand is isolated as a 1.3 kb HindIII/EcoRI fragment from the plasmid pKN100F19LC (NAGENESEQ: AAZ32784) and cloned into the corresponding cutting sites of the vector pBIN, thus producing the vector pBIN/F19LC (FIG. 1).

The deletion of the C-terminal lysine on the heavy chain of the F19 is carried out by PCR using the mutagenic primer F19HC-Lys rev gacgtctaga tcaacccgga gacagggaga ggc (SEQ ID NO:1) with a complementary sequence to the gene sequence which codes for the last amino acids of the heavy chain in the C-terminal region. Certainly, the codon of the C-terminal lysine is replaced by a stop codon. In addition, this is then followed by an XbaI restriction cutting site which is used for the later cloning. This mutagenic primer is used in conjunction with the primer F19 heavy4 atctgcaacg tgaatcacaa gc (SEQ ID NO:2), which has complementarity with another sequence located further upstream in the constant region of the heavy chain. The vector pBID/F19HC serves as a template for the PCR mutagenesis. The resulting PCR product of 757 by is digested with BmgBI (an endogenous cutting site located downstream of the primer position F19heavy4) and XbaI and the 547 by restriction fragment is used to replace the corresponding sequence region in the vector pBID/F19HC. This results in the vector pBID/IgG1-Lys, which codes for a heavy chain of the F19 antibody with a deleted C-terminal amino acid lysine (FIG. 1).

First of all a check is made by transient transfection of CHO-DG44 cells to find out whether the deleted C-terminal lysine, which is a highly conserved amino acid in all the IgG subtypes, has an essential significance for the expression or secretion of the IgG1 molecule. A co-transfection is carried out with the following plasmid combinations:
a) control plasmids pBID/F19HC and pBIN/F19LC, which code for the monoclonal antibody F19 in its wild-type configuration, i.e. including the C-terminal lysine on the heavy chain
b) pBID/IgG1-Lys and pBIN/F19LC, which code for an F19-antibody, the heavy chain of which comprises a C-terminal lysine deletion

10 Pools are transfected per combination, while equimolar amounts of the two plasmids are used in each co-transfection. After 48 h cultivation in a total volume of 3 mL the harvesting and determination of the IgG1-titre in the cell culture supernatant are carried out by ELISA. Differences in the transfection efficiency are corrected by co-transfection with an SEAP expression plasmid (addition of in each case 100 ng of plasmid-DNA per transfection mixture) and subsequent measurement of the SEAP activity.

Surprisingly it can be shown that the IgG1 molecule is expressed and secreted in CHO cells in spite of the deletion of the C-terminal lysine and the amounts of product are comparable with those of IgG1 wild-type transfected cells (FIG. 2).

For a stable transfection of CHO-DG44 cells, co-transfection is carried out with the same plasmid combinations as described above, producing 10 pools for each combination. As a negative control, 2 mock-transfected pools are also run, i.e. treated in the same way, but without the addition of DNA to the transfection mixture. The selection of stably transfected cells takes place two days after the transfection in HT-free medium with the addition of 400 μg/mL of G418. Once selection has taken place the IgG1 titre and the specific productivity of the cell pools is determined over a period of 3-4 passages (passaging rate 2-2-3 days). Surprisingly it is found that cells which express the lysine deletion variant of IgG1 achieve on average even 27% higher titres or 32% higher specific productivities than cells which express the IgG1 wild-type (FIG. 3). This production advantage of the lysine deletion variant is still obtained even when a DHFR-based gene amplification is induced in these cell pools by the addition of 100 nM MTX. The titres and specific productivities are on average 86% and 120% higher, respectively (FIG. 4).

### Example 2

### Cloning and Expression of IGG4 with C-Terminal Lysine Deletion

In order to express a monoclonal humanised IgG4 antibody (IgG4/kappa) the heavy chain is cloned as a 2.2 kb BamHI/SmaI fragment into the plasmid pBIDa digested with EcoRI (cutting site topped up by treatment with Klenow-DNA-polymerase) and BamHII, resulting in the plasmid pBIDa/IgG4 HC (FIG. 1. The light chain on the other hand is cloned as a 1.1 kb BamHI/EcoRI-fragment into the BamHI/EcoRI cutting sites of the plasmid pBINa, thus producing the plasmid pBIN8a/IgG4 LC (FIG. 1).

The deletion of the C-terminal lysine on the heavy chain of the IgG4 antibody is carried out by PCR using the mutagenic primer IgG4HC-Lys rev gacgtctaga tcaacccaga gacagggaga ggct (SEQ ID NO:3) with a sequence complementary to the sequence that codes for the last amino acids of the heavy chain in the C-terminal region. However, the codon of the C-terminal lysine is replaced by a stop codon. In addition, this is followed by a XbaI restriction cutting site, which is used for the later cloning. This mutagenic primer is used in conjunction with the primer HC for8 cccctgacct aagcccaccc (SEQ ID NO:4), which has complementarity with a sequence located further upstream in the constant region of the heavy chain. The vector pBIDa/IgG4 HC serves as a template for the PCR mutagenesis. The resulting PCR product of 1013 by is digested with BmgBI (an endogenous cutting site located downstream of the primer position HC for8) and XbaI and the 644 by restriction fragment is used to replace the corresponding sequence region in the vector pBIDa/IgG4 HC. This results in the vector pBIDa/IgG4-Lys, which codes for a heavy chain of the F19 antibody with a deleted C-terminal amino acid lysine (FIG. 1).

First of all a check is made by transient transfection of CHO-DG44 cells to find out whether the deleted C-terminal lysine, which is a highly conserved amino acid in all the IgG subtypes, has an essential significance for the expression or secretion of the molecule. A co-transfection is carried out with the following plasmid combinations:
a) control plasmids pBIDa/IgG4 HC and pBIN8a/IgG4 LC, which code for the monoclonal IgG4 antibody in its wild-type configuration, i.e. including the C-terminal lysine on the heavy chain
b) pBIDa/IgG4-Lys and pBIN8a/IgG4 LC, which code for a monoclonal IgG4 antibody, the heavy chain of which comprises a C-terminal lysine deletion

10 Pools are transfected per combination, while equimolar amounts of the two plasmids are used in each co-transfection. After 48 h cultivation in a total volume of 3 mL the harvesting and determination of the IgG4 titre in the cell culture supernatant are carried out by ELISA. Differences in the transfection efficiency are corrected by co-transfection with an SEAP expression plasmid (addition of in each case 100 ng of plasmid-DNA per transfection mixture) and subsequent measurement of the SEAP activity.

Surprisingly it can be shown that the IgG4 molecule is even produced rather better than the IgG4 wild-type in spite of the deletion of the C-terminal lysine in CHO cells (FIG. 5).

For a stable transfection of CHO-DG44 cells, co-transfection is carried out with the same plasmid combinations as described above, producing 10 pools for each combination. As a negative control, 2 mock-transfected pools are also run, i.e. treated in the same way, but without the addition of DNA to the transfection mixture. The selection of stably transfected cells takes place two days after the transfection in HT-free medium with the addition of 400 μg/mL of G418. Once selection has taken place, DHFR-based gene amplification is induced by the addition of 100 nM of MTX. The IgG4 titre and the specific productivity of the cell pools is determined over a period of 3-4 passages (passaging rate 2-2-3 days). In all, after the selection and amplification from the cells transfected with IgG4 wild-type, 4 stably expressing cell pools are obtained, and from the cells transfected with the lysine deletion variant, 6 stably expressing cell pools are obtained. Surprisingly it is found that cells which express the lysine deletion variant of IgG4 achieve on average even 63% higher titres or 70% higher specific productivities than cells which express the IgG4 wild-type (FIG. 6). This production advantage of the lysine deletion variant is still obtained even in the subsequent amplification step with 400 nM MTX. On average 53% higher titres and 66% higher specific productivities are obtained (FIG. 7).

### Example 3

### Cloning and Expression of IGG2, IGG3, FC Fusion Proteins and Other Biomolecules with C-Terminal Amino Acid Deletion

In order to delete the C-terminal lysine on the heavy chains of the antibody isotypes IgG2 and IgG3, PCR mutagenesis is used, as described earlier in Examples 1 and 2 for isotypes 1 and 4. In the same way C-terminal lysine deletions are also carried out on Fc fusion proteins (bivalent or bispecific), in which biomolecules such as cytokines, soluble receptors, etc., are components of an Fc fusion protein (examples: Alefacept, LFA-3-Fc, Etanercept TNFR-Fc).

It is conceivable to extend the concept of codon deletion of C-terminal amino acids to biomolecules such as e.g. erythropoietin (EPO) and Tissue Plasminogen Activator (tPA), in which proteolytic cleaving of the C-terminal arginine is known (M. A. Recny, H. A. Scoble and Y. Kim, J. Biol. Chem., 262 (1987) 17156-17163; Harris, R. J. (1995) Journal of Chromatography A, 705 (1), pp. 129-134). The prerequisite is that the biological activity is maintained or, as in the case of tPA, for example, proteolytic processing remains intact. In transient transfections first of all a test is carried out to discover whether the deletion of the C-terminal amino acid affects the expression and secretion. Then stable transfections are carried out and the specific productivities and titres of cell pools which express mutated proteins or wild-type proteins are compared with one another.

### Example 4

### Purification of IGG1 WT and IGG1-Lys

The working up is identical for isotype IgG1 or the WT and the lysine deletion variant. The protein A affinity chromatography (MabSelect, GE) is carried out according to the manufacturer's instructions.

The quantification of the product yield after protein A chromatography is carried out using protein A HPLC. The yields for both variants of the isotype IgG1 independently of the lysine codon deletion are over 90% (FIG. 8). The lysine deletion has no negative effect on the affinity chromatography or the product yield. The product heterogeneity with regard to the C-terminal lysine is determined both qualitatively in the isoelectric focusing (IEF) and also quantitatively by weak cationic exchange (WCX) (cf. FIGS. 9 and 11). In order to determine the charge heterogeneity caused by C-terminal lysine qualitatively using IEF, the antibodies are incubated with carboxypeptidase B. At 37° C., 10 μg of carboxypeptidase B are incubated in 100 μL at an antibody concentration of 1 mg/mL for 2 h. In FIG. 9 there is a reduction in the number of bands for the WT antibody (IgG1) after enzymatic cleaving with carboxypeptidase B (CpB).

The cation exchange chromatography (ProPac WCX-10/4×250 mm) is carried out with a flow rate of 1 mL/min and a gradient of 5-10% over 40 min (bufferA 20 mM MES-buffer pH 6.7; bufferB: 20 mM, 1M NaCl pH6.7). The column is charged with 40 μg antibody in each case. The WT and the -Lys variant are each analysed with or without enzymatic CpB treatment.

In the elution profile or overlay of the IgG1 WT the states of Lys1 and Lys2 are represented by the basic peaks 1 and 2. The proportion of product is ˜10%. The elution profile or overlay of the variant with lysine deletion shows very slight heterogeneity in the basic region. The proportion of product is less than 1% (FIG. 11).

### Example 5

### Purification of IGG4 WT and IGG4-Lys

For the isotype IgG4 or the WT and the lysine deletion variant the working up is identical to IgG1. The protein A affinity chromatography (MabSelect, GE) is carried out according to the manufacturer's instructions.

The quantification of the product yield after protein A chromatography is carried out using protein A HPLC. The yields for both variants of the isotype IgG4 independently of the lysine codon deletion are also over 90% (FIG. 8). For the isotype IgG4 and its lysine codon deletion, as in IgG1, it is confirmed that there is no negative effect on the affinity chromatography or the product yield. The product heterogeneity with regard to the C-terminal lysine is determined quantitatively by LC-MS (cf. FIGS. 10 and 12). In order to determine the C-terminal lysine distribution, the antibody samples are first reduced with DTT. Then the reduced light chain and the reduced heavy chain (HC 1-446 without Lys or HC1-447 with Lys) are separated by HPSEC separated and analysed in subsequent (in-line) ESI-TOF-MS. The distribution of the C-terminal lysine is based on the peak areas for the HC 1-446 or HC 1-447. The mass spectrogram of the heavy chain shows the mass shift caused by the lysine as a function of the glycosylation (G0, G1, G2) (FIG. 12). The product proportion of antibody molecules determined (IgG4 WT) with C-terminal lysines in the heavy chain (Lys1 and Lys2) is approx. 20% (FIG. 10).

### Example 6

### Thermal Stability

The determination of the thermal stability using intrinsic fluorescence (tryptophan) shows no influence on the part of the C-terminal lysine (IgG1 WT and -Lys Tₘ 69° C.; or IgG4 WT and -Lys 64° C.). The excitation wavelength is 295 nm. The particular emission spectrum is measured in 1° C. increments over a range from 25° C. to 85° C. The emission spectra are recorded over a wavelength range of from 300 nm to 450 nm. Other technical data: fluorescence spectrometer LS55 Perkin Elmer, slot width 4 nm for temperature measurement on the excitation and emission side/PT100 in the sample.

The protein concentrations were 0.1 mg/mL in PBS buffer. The investigation shows that the C-terminal lysine of the heavy antibody chain has no influence on the thermal stability of the antibody molecule.

Earlier investigations had already shown that the C-terminal lysine of the heavy antibody chain has no influence on the thermal stability of the antibody molecule (Liu et al. Immunol. Lett. 2006, 106 (2), 144-153).

1. Method of increasing the titre of a protein of interest of a cell, characterised in that
a. in a nucleic acid sequence which codes for the protein of interest, at least the codon which codes for the C-terminal amino acid is deleted,
b. the cell is transfected with a vector which contains the modified nucleic acid from a) and
c. the cell is cultivated under conditions that permit the production of the protein of interest.

2. Method according to claim 1, characterised in that the titre is increased by at least 10%, 20%, 50%, preferably 75% relative to the comparative value of the protein without the deletion of the C-terminal amino acid.

3. Method according to claim 1, characterised in that the specific productivity of the cell is increased by at least 10%, 20%, 50%, preferably 75% relative to the comparative value of the protein without the deletion of the C-terminal amino acid.

4. Method of producing an expression vector for the increased production of a protein of interest, characterised in that
a. in the nucleic acid sequence which codes for the protein of interest, at least the codon which codes for the C-terminal amino acid is deleted, and
b. the nucleic acid sequence from a) thus modified is inserted in an expression vector.

5. Method of producing a cell with an increased titre of a protein of interest, characterised in that
a. a group of cells is treated by a method according to claim 1 and
b. then single cell cloning is carried out.

6. Method for preparing of a protein of interest in a cell, characterised in that
a. a group of cells is treated by a method according to claim 1,
b. these cells are selected from a) in the presence of at least one selection pressure,
c. optionally a single cell cloning is carried out and
d. the protein of interest is obtained from the cells or the culture supernatant.

7. Method for preparing at least one protein of interest according to claim 6, characterised in that the cells used for the preparation are additionally subjected to a gene amplification step after step b) has been carried out.

8. Method according to claim 1, characterised in that the C-terminal amino acid is lysine (Lys) or arginine (Arg), preferably Lys.

9. Method according to claim 1, characterised in that the protein of interest is an antibody, an Fc fusion protein, EPO or tPA.

10. Method according to claim 1, characterised in that the protein of interest is a heavy chain of an antibody and the C-terminal amino acid is lysine (Lys).

11. Method according to claim 10, characterised in that the heavy chain of the antibody is of the type IgG1, IgG2, IgG3 or IgG4, preferably of the type IgG1, IgG2 or IgG4.

12. Method according to claim 1, characterised in that the protein of interest is a monoclonal, polyclonal, mammalian, murine, chimeric, humanised, primate or human antibody or an antibody fragment or derivative of a heavy chain of an immunoglobulin antibody or of a Fab, F(ab′)2, Fc, Fc-Fc fusion protein, Fv, single chain Fv, single domain Fv, tetravalent single chain Fv, disulphide-linked Fv, domain-deleted antibody, a minibody, diabody or a fusion polypeptide of one of the above-mentioned fragments with another peptide or polypeptide or an Fc-peptide fusion protein, an Fc-toxin fusion protein or a scaffold protein.

13. Method according to claim 1, characterised in that the cell is cultivated in suspension culture.

14. Method according to claim 1, characterised in that the cell is cultivated under serum-free conditions.

15. Method according to claim 1, characterised in that the cell is a eukaryotic cell, e.g. from yeast, plants, worms, insects, birds, fish, reptiles or mammals.

16. Method according to claim 15, characterised in that the cell is a mammalian cell.

17. Method according to claim 16, characterised in that the cell is a CHO cell, preferably a CHO DG44 cell.

18. Expression vectors with increased expression of a gene of interest which may be generated according to a method according to claim 4.

19. Cell which may be generated by a method according to claim 5.

20. Method for the production and purification of a protein of interest, characterised in that
a. in a nucleic acid sequence which codes for the protein of interest, at least the codon which codes for the C-terminal amino acid is deleted, and
b. the resulting protein of interest has decreased heterogeneity compared with the protein without the deletion of the C-terminal amino acid.

21. Method according to claim 20, characterised in that the C-terminal amino acid is lysine (Lys) or arginine (Arg), preferably Lys.

22. Method according to claim 20, characterised in that the protein of interest is an antibody, an Fc fusion protein, EPO or tPA.

23. Method according to claim 20, characterised in that the protein of interest is a heavy chain of an antibody and the C-terminal amino acid is lysine (Lys).

24. Method according to claim 23, characterised in that the heavy chain of the antibody is of the type IgG1, IgG2, IgG3 or IgG4, preferably of the type IgG1, IgG2 or IgG4.

25. Method according to claim 20, characterised in that during the purification of the protein of interest a lower salt concentration is used compared with the purification of a protein without the deletion of the C-terminal amino acid.


SEQUENCE LISTING (4 sequences)

SEQ ID NO: 1 - DNA, 33 bp, Artificial
  Primer F19HC-Lys rev
gacgtctagatcaacccggagacagggagaggc

SEQ ID NO: 2 - DNA, 22 bp, Artificial
  Primer F19 heavy 4
atctgcaacgtgaatcacaagc

SEQ ID NO: 3 - DNA, 34 bp, Artificial
  Primer IgG4HC-Lys rev
gacgtctagatcaacccagagacagggagaggct

SEQ ID NO: 4 - DNA, 20 bp, Artificial
  Primer HC for8
cccctgacctaagcccaccc